(19) United States
(12) Patent Application Publication
(10) Pub. No.: US 2025/0170110 A1
(43) Pub. Date: May 29, 2025

(54) LYMPHATIC TARGETED ANTI-COAGULANT FOR THE PREVENTION OF LYMPHATIC THROMBOSIS

(71) Applicant: THE TRUSTEES OF THE UNIVERSITY OF PENNSYLVANIA, Philadelphia, PA (US)

(72) Inventors: Mark L. KAHN, Bryn Mawr, PA (US); John D. WELSH, Philadelphia, PA (US); Xiaowen CHEN, Philadelphia, PA (US); Jacob MYERSON, Media, PA (US)

(73) Assignee: THE TRUSTEES OF THE UNIVERSITY OF PENNSYLVANIA, Philadelphia, PA (US)

(21) Appl. No.: 19/040,198

(22) Filed: Jan. 29, 2025

Related U.S. Application Data
(63) Continuation of application No. PCT/US2023/028994, filed on Jul. 28, 2023.
(60) Provisional application No. 63/393,409, filed on Jul. 29, 2022.

(51) Int. Cl. A61K 31/437; A61K 9/00; A61K 47/54

(52) U.S. Cl. CPC A61K 31/437; A61K 9/0053; A61K 47/543

(57) ABSTRACT

Novel methods, compositions, and kits for using lymph targeted antithrombotic (LTAT) molecules are provided herein. The LTAT molecules can be used for treating or preventing of lymphatic thrombosis resulting from infection or inflammation, especially in the gut.

### CROSS-REFERENCE TO RELATED APPLICATIONS

This application is a continuation of International Patent Application No. PCT/US2023/028994, filed Jul. 28, 2023, which claims priority U.S. Provisional Patent Application Ser. No. 63/393,409, filed on Jul. 29, 2022, the contents of which are incorporated by reference herein in their entireties.

### GRANT INFORMATION

This invention was made with government support under DK123528 awarded by the National Institutes of Health. The government has certain rights in the invention.

### 1. FIELD

The present disclosed subject matter relates gut lymph targeted antithrombotic molecules for the treatment or prevention of gut lymphatic thrombosis. The disclosed subject matter further provides methods, compositions, and kits for treating inflammation, coagulation, and cellular aggregation that impairs lymphatic function in the gut.

### 2. BACKGROUND

The lymphatic system is a vascular system that serves to collect and return interstitial fluid to the blood stream, transport immune cells, and transport fat absorbed from the gut. Interstitial fluid is generated by the leakage of fluid from the blood stream into tissue where it is then collected by the lymphatic system for return to the blood stream. It has long been known that lymph fluid contains high levels of the molecular components of the clotting cascade, and that lymph fluid can form fibrin clots in vitro and ex vivo models of clot initiation. Lymphatic clots can form in association with infections that involve lymphatic vessels, but such lymphatic thrombi are rare and whether they truly arise independently of blood and blood-derived cells is not clear.

The gut lymphatic vascular network drains interstitial fluid and immune cells from the stomach, small intestine, and colon. The small intestine lymphatics also play a specialized role in the absorption of fat from the gut through the transport of chylomicrons formed by the gut epithelium from dietary lipids. These chylomicrons are transported along with lymph fluid from the gut into the collecting lymphatic system and are delivered to the blood system through the lympho-venous junction. The gut lymphatics play a key role in transporting gut immune cells that are constantly being challenged by the constant exposure of the gut tissue to endogenous and exogenous bacteria and other challenges. The immune monitoring of the gut can require patent lymphatic vessels to transport and remove activated immune cells and regulate the inflammatory duration and intensity.

### 3. SUMMARY

The purpose and advantages of the disclosed subject matter will be set forth in and are apparent from the description that follows, as well as will be learned by practice of the disclosed subject matter. Additional advantages of the disclosed subject matter will be realized and attained by techniques particularly pointed out in the written description and claims hereof, as well as from the appended drawings.

To achieve these and other advantages and in accordance with the purpose of the disclosed subject matter, as embodied and broadly described, the disclosed subject matter includes lymph targeted antithrombotic (LTAT) molecules and methods for using said molecules for treating inflammatory and thrombotic conditions affecting the gut lymphatic system. In addition to methods of treatment, the disclosed subject matter further provides for pharmaceutical compositions and kits comprising said molecules, together with a suitable pharmaceutical carrier.

In a first aspect, the present disclosure provides for a lipid conjugate, wherein the lipid conjugate comprises one or more lymphatic condition, disease or disorder treatment active molecules; and a lipid or lipid-like molecule. In certain embodiments the lipid conjugate further comprises a linker region. In certain embodiments the lipid conjugate further comprises lipid head group.

In certain embodiments, the one or more lymphatic condition, disease or disorder treatment active molecules is an anticoagulant molecule. In certain embodiments, the anticoagulant molecule is an anti-thrombin, anti-Xa molecule, or thrombolytic agent. In certain embodiments, the anticoagulant molecule is a anti-thrombin, anti-Xa molecule, or thrombolytic agent. In certain embodiments, the anticoagulant molecule blocks thrombin or Xa enzymatic activity.

In certain embodiments, the anti-thrombin molecule is hirudin, bivalirudin, ximelagatran, a derivative of dabigatran, or a derivative of tripeptide-type thrombin inhibitors. In certain embodiments, the anti-Xa molecule is selected from the group consisting of Edoxaban, Rivaroxaban, Apixaban, or combinations thereof.

In certain embodiments, lipid comprises long chain fatty acids or monoglycerides.

In certain embodiments, lymphatic condition, disease or disorder active molecule is selected from an active agent that can treat the lymphatic condition, disease or disorder selected from the group consisting of sepsis, necrotizing enterocolitis, autoimmune diseases, Crohn's disease, celiac disease, ulcerative colitis, rheumatoid arthritis, cardiovascular disease, bacterial infections, viral infections, viral hepatitis (including hepatitis C virus hepatitis), alcoholic hepatitis, insulin resistance in adipocytes, pancreatitis, metabolic syndrome, a trauma induced inflammation, an acute respiratory distress syndrome (ARDS), a COVID-19-induced systemic inflammation, an organ rejection after transplantation, amyloidosis, lymphangitis, obesity, primary or secondary lymphedema, congenital lymphatic insufficiency, lymphatic vessel aplasia, inflammatory bowel disease (including Crohn's disease and Ulcerative Colitis), chronic granulomatous disease (CGD), lymphatic malignancies (including but not limited, Hodgkin's Disease, non-Hodgkin's lymphoma, and Castleman Disease), Milroy's disease, Meige's disease, elephantiasis, disorders of the lymphatic system arising secondarily to tissue damage, e.g., an infarction, injury from surgery, organ or tissue transplant, radiation therapy, chemotherapy, and occlusion or blockage (full or partial) of lymph vesselsm non-lymphatic malignancies, colorectal cancer, liver cancer, stomach cancers, pancreatic cancer, sepsis, necrotizing enterocolitis, autoimmune diseases, and Castleman Disease), Milroy's disease, Meige's disease, elephantiasis, disorders of the lymphatic system arising secondarily to tissue damage, e.g., an infarction, injury from surgery, organ or tissue transplant, radiation therapy, chemotherapy, and occlusion or blockage (full or partial) of lymph vessels.

In certain embodiments, present disclosure provides a method of treating gut lymphatic thrombosis, comprising administering to a subject in need of such treatment, an effective amount of the lipid conjugate, wherein administering an effective amount of lipid based conjugate preserves hemostasis within the subject.

In certain embodiments, lymphatic thrombosis is associated with an infection or inflammation in at least the gut.

In certain embodiments, the lipid conjugate is selectively packaged with chylomicrons and transported from the gut in lymph. In certain embodiments, the lipid conjugate is not readily absorbed into the blood stream.

In certain embodiments, the lipid conjugate is in the form of a tablet, a capsule, a sachet, a suppository, a liquid, an oil, or combination thereof.

In certain embodiments, the lipid conjugate is administered orally.

In certain embodiments, the lipid conjugate is a free lipid within an oil solution, micelle, liposomes, or solid lipid nanoparticles.

In certain embodiments, the present disclosure provides a method of preventing lymph clot formation using a lipid conjugate.

In certain embodiments, the present disclosure provides a method for reducing inflammation of the gut using a lipid conjugate.

In certain embodiments, the present disclosure provides a method for treating infections of the gut using a lipid conjugate.

In certain embodiments, the present disclosure provides a pharmaceutical composition comprising a lipid conjugate.

In certain embodiments, the present disclosure provides a kit comprising a lipid conjugate.

In a first aspect, the present disclosure provides a method for treating lymphatic thrombosis, including administering to a subject in need of such treatment, an effective amount of a lymph targeted antithrombotic molecule (LTAT). In certain embodiments, the lymphatic thrombosis is associated with an infection or inflammation at least in the gut.

In certain embodiments, the LTAT molecule is selectively packaged with chylomicrons and transported from the gut in lymph rather than blood, unlike a non-lipid based antithrombotic molecule. In certain embodiments, the LTAT molecule is not readily absorbed into the blood stream compared to a non-lipid based antithrombotic molecule. In certain embodiments, the LTAT molecule comprises a lipid or lipid-like molecule conjugated to an active agent, such as an anticoagulant.

In certain embodiments, the LTAT molecule further comprises a linker region. In certain embodiments, the LTAT molecule further comprises a lipid head group.

In certain embodiments, the anticoagulant molecule is a anti-thrombin, anti-Xa molecule, or thrombolytic agent. In certain embodiments the anticoagulant molecule blocks thrombin or Xa enzymatic activity.

In certain embodiment, the anti-thrombin molecule is hirudin, bivalirudin, ximelagatran, a derivative of dabigatran, or a derivative of tripeptide-type thrombin inhibitors.

In certain embodiments the anti-Xa molecule is selected from Edoxaban, Rivaroxaban, Apixaban, or combinations thereof.

In certain embodiments, the lipid comprises long chain fatty acids or monoglycerides.

In certain embodiments, the LTAT is in the form of a tablet, a capsule, a sachet, a suppository, a liquid, an oil, or combination thereof.

In certain embodiments, the LTAT molecule is administered orally. In certain embodiments, the LTAT molecule is a free lipid within an oil solution, micelle, liposomes, or solid lipid nanoparticles.

In another aspect, the present disclosure provides a method of preventing, reducing or treating lymph clot formation using a method described herein. In certain embodiments, the present disclosure provides a method for reducing inflammation of the gut. In certain embodiments, the present disclosure provides a method for treating infections of the gut.

In another aspect, the present disclosure provides a pharmaceutical composition for performing the methods described herein.

In another aspect, the present disclosure provides a kit for performing the methods described herein.

The present disclosure further provides a bioconjugate, wherein the bioconjugate comprises an active agent, such as an anticoagulant molecule and a lipid or lipid-like molecule. In certain embodiments, the bioconjugate further comprises a linker region. In certain embodiments, the bioconjugate further comprises a lipid head group.

### 4. BRIEF DESCRIPTION OF THE DRAWINGS

FIGS. 1A and 1B show mouse lymphatic endothelial cell expression of antithrombotic proteins. FIG. 1A shows representative images of immunohistological staining of mouse tissue for PROX1 expression and EPCR. FIG. 1B shows a representative immunohistological staining of mouse tissue for PROX1 and thrombomodulin (THBD). Lymphatic vessels (L) and blood vessels (BV) are indicated for reference.

FIGS. 2A and 2B show a schematic of PAR1-Tango allele that reports thrombin activity in vivo in mice. FIG. 2A shows the lack of reporter activity when the PAR1 receptor is not exposed to thrombin, while FIG. 2B demonstrates activation of green fluorescent protein (GFP) expression in the presence of thrombin.

FIGS. 3A and 3B show PAR1-Tango activity in gut lymphatics. FIG. 3A shows PAR1-Tango activity is not in the lymphatic vessels in the lung, heart or skin. FIG. 3B shows PAR1-Tango activity for gut lymphatics. Lymphatic vessels are marked by staining for VEGFR3 or LYVE1. The LYVE1-positive vessels in the liver shown in FIG. 3A are sinusoidal blood vessels.

FIGS. 4A-4E show PAR1-Tango activity arises in coordination with the gut microbiome and can be reversed by antibiotics. FIG. 4A demonstrates that nuclei in LYVE1+ (lymphatic vessel endothelial hyaluronan receptor 1) lymphatic vessels are negative for GFP at P0 and P7 and begin to show GFP expression at P14 and then strong GFP expression at P21. FIG. 4B shows quantification of GFP positive cells over time. LYVE1-negative, GFP-positive cells are likely macrophages in the neonatal intestine. FIG. 4C shows a schematic representation of an example design and timeline for accessing gut microbiome in response to antibiotic treatment. FIG. 4D shows the inhibitory effect of neonatal antibiotic (“ABX”) treatment on PAR1-Tango reporter activity and is quantified in FIG. 4E, confirming that thrombin generation is linked to bacterial colonization of the gut. These data connect bacteria in the gut lumen to thrombin activity in the gut lymphatic lumen.

FIGS. 5A-5D show that bacterial infections create lymphatic clots. FIG. 5A shows the experimental design in which mature mice are exposed to Salmonella or Shigella orally through gavage. FIG. 5B demonstrates fibrin clots in LYVE1-positive lymphatic vessels in mice infected with Salmonella and Yersinia. FIG. 5C indicates that approximately 20% of gut lymphatic vessels were thrombosed after Salmonella infection. In FIG. 5D mice were gavaged with GFP-expressing Salmonella bacteria that could then be identified within fibrin clots in the gut lymphatic vessels. P-values are indicated by asterisks as follows: **p<0.01.

FIGS. 6A-6E show chronic small intestine inflammation causes lymphatic clot formation. FIG. 6A shows a schematic of the mouse dosing plan using 2.5% DSS to induce bowel inflammation over 7 days. FIG. 6B shows representative images of control and DSS treated mouse intestines stained for fibrin and VegFR3 a marker of lymphatic vessels. FIG. 6C shows quantification of the average percentage of fibrin positive lymphatic vessels showing the mean and standard deviation. FIG. 6D shows representative images of human gut samples from healthy and Crohn's disease subjects showing fibrin(ogen) staining and Lyve1 staining, a marker of lymphatic vessels. FIG. 6E shows quantification of the mean and standard deviation of the two groups. P-values are indicated by asterisks as follows: ***p<0.001, **p<0.01.

FIGS. 7A-7C show oral delivery of warfarin prevents lymphatic clots in mouse model of lymphothrombosis. FIG. 7A shows a schematic outlining the experimental design where mice with an inducible knockout allele of thrombomodulin were dosed with tamoxifen to induce deletion of thrombomodulin expression and were simultaneously given warfarin or vehicle treatment in their water. FIG. 7B shows representative images from the gut lymphatics of the experimental mice three weeks after first induction showing fibrin and Lyve1 as a marker of lymphatic vessels. FIG. 7C shows quantitative data showing the average and standard deviation of the percentage of fibrin positive lymphatic vessels after 3 weeks. P-values are indicated by asterisks as follows: ***p<0.001.

FIGS. 8A-8D show lymph targeted antithrombotic (LTAT) molecular architecture and design. FIG. 8A shows a schematic depicting the general architecture of an LTAT molecule with an antithrombotic molecule attached to a lipid head group via a flexible linker. The LTAT molecule could be delivered as a free lipid or as shown in FIG. 8B as a micelle or liposome with other lipids. FIG. 8C shows molecular structures of 4 direct oral anticoagulants. FIG. 8D shows two sample LTAT molecular designs showing either dabigatran or rivaroxaban chemically linked to lipids.

FIGS. 9A-9E illustrate the process of creating anticoagulant lipid-based nanoparticles. FIG. 9A depicts representative lipid formulations for constructing base liposomes. FIG. 9B illustrates the method of attaching anticoagulant molecules to the lipid base, resulting in the formation of an anticoagulant-lipid nanoparticle (NPs). Specifically, the thrombin inhibitor PPACK, functionalized with dibenzocyclooctyne, reacts with an azide group present on the liposome surface. FIG. 9C presents the comparison between the activity of the PPACK nanoparticle and free-isolated PPACK, utilizing a chromogenic substrate assay to measure thrombin activity. FIGS. 9C-D demonstrate the ability to trace PPACK NPs in chyle. FIG. 9D shows a representative fluorescent image of PPACK NPs after doping in drawn chyle. FIG. 9E shows the size distribution of PPACK NPs doped in chyle.

FIGS. 10A-10D demonstrate the inhibitory effect of oral PPACK NP treatment on thrombin in lymph. FIG. 10A demonstrates that thrombin activity is inhibited in chyle from mice treated with oral PPACK NPs, as evaluated using a thrombin-chromozym TH reaction assay. FIG. 10B depicts the quantification of thrombin activity in lymph from mice treated with oral PPACK NPs, expressed as the percent thrombin activity relative to sham-treated chyle. FIG. 10C depicts a dose-response curve for thrombin inhibition by lymph from mice treated with oral PPACK NPs, examining the effect of varying amounts of chyle containing PPACK NPs on thrombin activity. The curve reveals a 1 U/mL thrombin inhibition IC50 at 0.4 μL chyle. FIG. 10D shows depicts a dose-response curve examining thrombin inhibition by PPACK NPs prior to injection, revealing an IC50 of 0.03 μL. Comparing FIG. 10D to 10C indicates the concentration of PPACK-lipids in the chyle from mice treated with oral PPACK NPs.

FIGS. 11A-11C depict the tracking of fluorescent lipids, co-formulated in PPACK NPs, in chyle, and demonstrate that lipids from oral PPACK NPs are transported to chyle and incorporated in chylomicrons. FIG. 11A shows sizing and concentration measurements of nanoparticles, including chylomicrons, in chyle containing fluorescently labeled lipids. FIG. 11B shows the size distribution of all NPs, including chylomicrons, found in chyle from mice treated with oral PPACK NPs, compared to sham-treated mice. FIG. 11C shows the mean nanoparticle size of nanoparticles, including chylomicrons, found in chyle from mice treated with oral PPACK NPs, compared to sham-treated mice. FIG. 11D shows the total concentration of nanoparticles, including chylomicrons, found in chyle from mice treated with oral PPACK NPs, compared to sham-treated mice.

FIG. 12 illustrates the process of radiolabeling anticoagulant lipid-based nanoparticles.

FIGS. 13A-13D depict the pharmacokinetics of ¹¹¹In labeled lipids, co-formulated in PPACK NPs, in mice. Data was obtained over the course of 24 hours post-gavage. FIG. 13A shows the biodistribution of ¹¹¹In labeled lipids from PPACK NPs along the gastrointestinal (GI) tract. FIG. 13B shows the concentration of ¹¹¹In found in non-GI organs, including heart, lung, liver, spleen, kidney, and brain. FIG. 13C shows the concentration of ¹¹¹In found in various tissues of the GI tract. FIG. 13D shows the concentration of ¹¹¹In found in chyle, blood and urine.

FIGS. 14A-14C show the pharmacokinetics of PPACK-lipid-induced thrombin inhibition in chyle and plasma. FIG. 14A depicts a dose-response curve showing thrombin inhibition by PPACK-NPs at the concentration prepared prior to gavage, revealing an IC50 of 0.0085 μL. FIG. 14B shows that thrombin is inhibited in chyle from mice treated with oral PPACK NPs but thrombin is not inhibited in plasma from mice treated with oral PPACK NPs, as evaluated using a thrombin-chromozym TH reaction assay. FIG. 14C shows quantification of PPACK NP-induced thrombin inhibition in chyle and plasma, expressed as percent inhibition of thrombin activity relative to that in sham-treated chyle or untreated plasma.

FIGS. 15A-15F demonstrate the impact of administering PPACK NPs via gavage on the complete blood count. Mice were subjected to blood tests at different time points within a 24-hour period following the gavage of PPACK NPs. FIG. 15A presents the cell counts of total white blood cells (WBC), lymphocytes (LYM), monocytes (MON), and neutrophils (NEU). FIG. 15B shows the size variation of white blood cells over the 24-hour period after the post-gavage administration of PPACK NPs. FIG. 15C shows blood test results for red blood cells and hemoglobin. FIG. 15D shows the changes in red cell size following the gavage of PPACK NPs. FIG. 15E shows blood test results for platelets. FIG. 15F shows the changes in platelet size following gavage of PPACK NPs.

FIGS. 16A and 16B illustrate alternative approaches for developing anticoagulant lipid conjugates. FIG. 16A illustrates the modification process of the FXa inhibitor apixaban for lipid conjugation. FIG. 16B shows Factor Xa inhibitors DX-9065a and YM-60828 having structures amenable for covalent linkage to lipid head groups.

FIG. 17 illustrates that the FXa inhibitor apixaban, when modified with a linker for conjugation to lipids, retains its ability to inhibit FXa.

### 5. DETAILED DESCRIPTION

The present disclosure relates to method, compositions and kits for preventing lymphatic thrombosis in the gut without disrupting hemostasis within the treated individual. For purposes of clarity of disclosure, but not by way of limitation, the detailed description of the presently disclosed subject matter is divided into the following subsections:
5.1. Definitions;
5.2 Disorders;
5.3 Lipid conjugates
5.4. Methods of use;
5.5. Compositions; and
5.6. Kits.

### 5.1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

The use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s)” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and still up to 1% of a given value. Alternatively, particularly with respect to biological systems or processes, the term can mean within an order of magnitude, within 5-fold, and within 2-fold, of a value.

The term “cell” refers to any suitable cell for use in the present disclosure, e.g., eukaryotic cells. For example, but not by way of limitation, suitable eukaryotic cells include animal cells, e.g., mammalian cells. In certain embodiments, suitable cells are cultured cells. In certain embodiments, suitable cells are host cells, recombinant cells, and recombinant host cells. In certain embodiments, suitable cells are cell lines obtained or derived from mammalian tissues which can grow and survive when placed in media containing appropriate nutrients and/or growth factors.

The terms “expression” or “expresses,” as used herein, refer to transcription and translation occurring within a cell, e.g., mammalian cell. In certain embodiments, the level of expression of a gene and/or nucleic acid in a cell can be determined on the basis of either the amount of corresponding mRNA that is present in the cell or the amount of the protein encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

The term “thrombosis” in this document refers to a thrombus, or clot, that forms inside a lymphatic vessel, thereby obstructing the flow of lymph. The term encompasses lymphatic vessels are healthy but exposed to high thrombin due to the gut bacterial and immune cell environment, as well as damaged or diseased, edema, fibrosis, immune disorders, nutritional failure, and other conditions that may occur within the lymphatic system. Significantly, a lymphatic thrombus is primarily composed of a fibrin meshwork and does not contain prothrombotic cells such as platelets and neutrophils that circulate in blood but not in lymph. This term is distinct from a “blood clot” that forms within the blood vascular space. Both are stimulated by thrombin proteolysis of fibrinogen to form cross-linked fibrin, but blood clots contain and are created by platelets and neutrophils that do not circulate in lymph.

The terms “disease, disorder or condition” refers to a disease, disorder or condition that a patient has been diagnosed with or is suspected of having, particularly a disease, disorder or condition associated with lymphatic thrombosis. A disease, disorder or condition encompasses, without limitation thereto, pathogenic infections, inflammation associated conditions, side-effects associated with medications or treatments, as well as idiopathic conditions characterized by symptoms that include inflammation.

In some contexts, “lymphatic condition, disease or disorder” is intended to include all disorders characterized by insufficient or abnormal lymphatic function, including but not limited to, viral or bacterial infection, wounding, cancer, amyloidosis, sporadic cases, lymphangitis, obesity, primary or secondary lymphedema, congenital lymphatic insufficiency, lymphatic vessel aplasia, cardiovascular disease, heart disease, inflammatory bowel disease, ulcerative colitis, Crohn's disease, chronic granulomatous disease (CGD), lymphatic malignancies (including but not limited to Crohn's disease, Hodgkin's Disease, non-Hodgkin's lymphoma, and Castleman Disease), Milroy's disease, Meige's disease, elephantiasis, disorders of the lymphatic system arising secondarily to tissue damage, e.g., an infarction, injury from surgery, organ or tissue transplant, radiation therapy, chemotherapy, and occlusion or blockage (full or partial) of lymph vessels.

In some contexts, the term “lymphatic endothelial cell” (also referred to as a LEC) refers to endothelial cells that line lymph vessels and that are related to, but distinct from, those endothelial cells that line blood vessels which are referred to as “blood endothelial cells”.

The term ‘effective amount” refers to an amount of an active ingredient that is effective to relieve or reduce to some extent one or more of the symptoms of the disease in need of treatment, or to retard initiation of clinical markers or symptoms of a disease in need of prevention, when the compound is administered. Thus, an effective amount refers to an amount of the active ingredient which exhibit effects such as (i) reversing the rate of progress of a disease; (ii) inhibiting to some extent further progress of the disease; and/or, (iii) relieving to some extent (or eliminating) one or more symptoms associated with the disease. The effective amount may be empirically determined by experimenting with the compounds concerned in known in vivo and in vitro model systems for a disease in need of treatment. The context in which the phrase “effective amount” is used may indicate a particular desired effect. For example, “an amount of an LTAT molecule effective to prevent or treat a lymphatic thrombosis” and similar phrases refer to an amount of LTAT molecule that, when administered to a subject, will cause a measurable improvement in the subject's lymphatic thrombotic state. Unlike anticoagulation in the blood system, an effective LTAT cannot be measured by laboratory assessment of established clotting parameters because those are all based on blood and blood plasma. An effective amount may vary according to the weight, sex, age and medical history of the individual, as well as the severity of the patient's condition(s), the type of disease(s) and mode of administration. An effective amount may be readily determined using routine experimentation, e.g., by titration (administration of increasing dosages until an effective dosage is found) and/or by reference to amounts that were effective for prior patients.

A subject can be a human or a non-human animal, for example, but not by limitation, a non-human primate, a dog, a cat, a horse, a rodent, a cow, a goat, a rabbit, etc.

As used herein, the term or “conjugate” refers to two or more components joined by a covalent bond, in which at least one of the components is a biomolecule such as an enzyme, protein or antibody. For example, the presently disclosed conjugates can include lipids or lipid-like molecules covalently joined to anticoagulant molecules.

The term, “carrier,” refers to a diluent, adjuvant, excipient or vehicle with which the therapeutic is administered. Such physiological carriers can be sterile liquids, such as water and oils, including those of petroleum, animal, vegetable or synthetic origin, such as peanut oil, soybean oil, mineral oil, sesame oil and the like. Water is a suitable carrier when the pharmaceutical composition is administered intravenously. Saline solutions and aqueous dextrose and glycerol solutions also can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents.

The term “agent,” as used herein, means a substance that produces or is capable of producing an effect and would include, but is not limited to, chemicals, pharmaceuticals, biologics, small organic molecules, antibodies, nucleic acids, peptides and proteins.

As used herein, the term “inhibitor” refers to a compound or molecule (e.g., small molecule, peptide, peptidomimetic, natural compound, siRNA, anti-sense nucleic acid, aptamer, or antibody) that interferes with (e.g., reduces, prevents, decreases, suppresses, eliminates or blocks) the signaling function of a protein or pathway. An inhibitor can be any compound or molecule that changes any activity of a protein (signaling molecule, any molecule involved with the named signaling molecule or a named associated molecule), such as RNF167, or interferes with the interaction of a protein, e.g., RNF167, with signaling partners. Inhibitors also include molecules that indirectly regulate the biological activity of a named protein, e.g., RNF167, by intercepting upstream signaling molecules.

### 5.2 Disorders

In certain embodiments, the lipid conjugates, methods, compositions, and kits provided herein can be used to treat any condition or disease/disorder (“disorder”) involving gut lymphatic thrombosis, including, but not limited to insufficient or abnormal lymphatic function, including but not limited to, infection, wounding, cancer, and inflammation.

In certain embodiments, the lymphatic disorder can be associated with any condition that causes gut inflammation, including sepsis, necrotizing enterocolitis, autoimmune diseases, Crohn's disease, celiac disease, ulcerative colitis, rheumatoid arthritis, cardiovascular disease, bacterial infections, viral infections, viral hepatitis (including hepatitis C virus hepatitis), alcoholic hepatitis, insulin resistance in adipocytes, pancreatitis, metabolic syndrome, a trauma induced inflammation, an acute respiratory distress syndrome (ARDS), a COVID-19-induced systemic inflammation, an organ rejection after transplantation, amyloidosis, lymphangitis, obesity, primary or secondary lymphedema, congenital lymphatic insufficiency, lymphatic vessel aplasia, inflammatory bowel disease, chronic granulomatous disease (CGD), lymphatic malignancies (including but not limited, Hodgkin's Disease, non-Hodgkin's lymphoma, and Castleman Disease), Milroy's disease, Meige's disease, elephantiasis, disorders of the lymphatic system arising secondarily to tissue damage, e.g., an infarction, injury from surgery, organ or tissue transplant, radiation therapy, chemotherapy, and occlusion or blockage (full or partial) of lymph vessels. In certain embodiments, lymphatic condition can also include non-lymphatic malignancies that nonetheless affect the lymphatic system, including, but not limited to, colorectal cancer, liver cancer, stomach cancers, pancreatic cancer. Insufficient or abnormal lymphatic function can result, e.g., from a defect in or deficiency of any component of the lymphatic system, including valves, capillaries, ducts, etc. Repairing or modulating expansion of any of these and any other lymphatic system components are contemplated using the methods described herein.

### 5.3 Lipid Conjugates

In certain embodiments, the present disclosure involves lipid conjugates. In certain embodiments, the present disclosure involves a lipid or lipid-like component conjugated to an active agent, such as an anticoagulant molecule. In certain non-limiting embodiments, the anticoagulant molecule is an antithrombotic molecule. In certain embodiments, the lipid antithrombotic molecule is a lymph-targeted antithrombotic (LTAT) molecule. In certain embodiments, the lipid anticoagulant molecule is targeted to the gut lymphatic system. The lipid anticoagulant can be delivered as free lipids within an oil solution, or as micelles, liposomes, or solid lipid nanoparticles to enhance absorption and availability of the anticoagulant in the lymphatic system. The conjugation between the lipid or lipid-like component and anticoagulant molecules prevents separation of the lipid and anticoagulant components upon administration, ensuring their integrity and effectiveness for targeted delivery in the lymphatic system. The lipid anticoagulant molecules can be used for targeted anticoagulant treatment focused on the lymphatic system.

Non-limiting examples of lipid or lipid-based molecules include dipalmitoylphosphatidylcholine (DPPC), cholesterol, distearoylphosphatidylethanolamine (DSPE), phosphatidylcholine (PC) and phosphatidylethanolamine (PE).

In certain embodiments, the lipid comprises long chain fatty acids or monoglycerides. Non-limiting examples of long chain fatty acids include palmitic acid (C16:0); stearic acid (C18:0); oleic acid (C18:1), linoleic acid (C18:2), alpha-linolenic acid (C18:3). Non-limiting examples of monoglycerides include monoolein, monostearin, monoglyceride, and monopalmitin

In certain non-limiting embodiments, the anticoagulant is not orally available to the blood stream when part of the lipid structure or on its own.

In certain non-limiting embodiments, the anticoagulant is resistant to proteases of the digestive system.

In certain non-limiting embodiments, the anticoagulant molecule is a anti-thrombin, anti-Xa molecule, or thrombolytic agent. The anticoagulant molecule can block thrombin or Xa enzymatic activity.

Non-limiting examples of anti-thrombin molecules include hirudin, bivalirudin, ximelagatran, a derivative of dabigatran, and a derivative of tripeptide-type thrombin inhibitors.

Non-limiting examples of anti-Xa molecules include Edoxaban, Rivaroxaban, Apixaban, DX-9065a and YM-60828

In certain non-limiting embodiments, the lipid or lipid-like molecule is conjugated to the anticoagulant molecules by reacting the lipid or lipid-like molecule with the anticoagulant molecule through click chemistry, esterification reaction, amidation reaction, or another conjugating reaction.

In certain non-limiting embodiments, the anticoagulant is covalently linked with a lipid head group, wherein the linkage point is sufficiently distanced from the enzyme interface so as not to reduce the efficacy of the inhibition. Non-limiting examples of covalent linkages terminal sulfhydryls, acids, hydroxyls, esters, aldehydes, and amines, among others.

In certain non-limiting embodiments, the lipid or lipid-based molecule comprises a reactive moiety. In certain non-limiting embodiments, the anticoagulant molecule comprises a reactive moiety. In certain non-limiting embodiments, the reactive moiety is a ligation moiety, such as, but not limited to trans-cyclooctene, tetrazine, cyclooctyne, alkyne, or azide, alkene, tetrazole, photo-DIBO or cyclopropenones.

In certain non-limiting embodiments, the conjugation between the lipid or lipid-like molecule with the anticoagulant molecule can be prepared by a different reaction scheme, as known in the art. Examples of suitable reactions include, but are not limited to, Diels-Alder reactions, azide-alkyne based click reactions (e.g., Cu(I) catalyzed azide-alkyne cycloaddition and metal-free azide-alkyne cycloadditions), Staudinger ligation, thiol-maleimide addition, oxime ligation, and thiol-ene reactions.

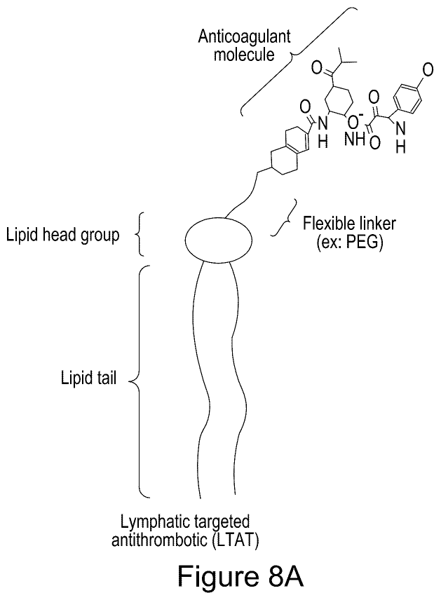

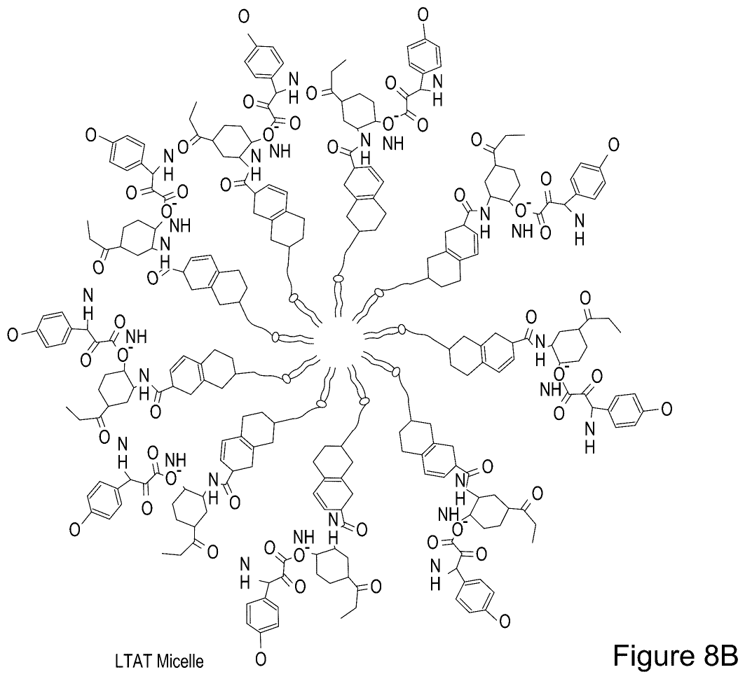

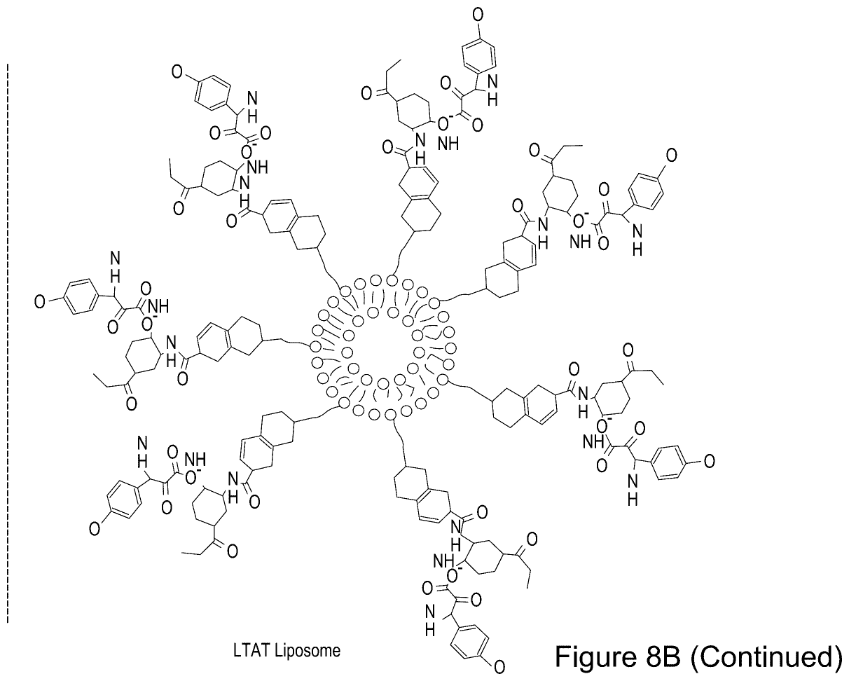

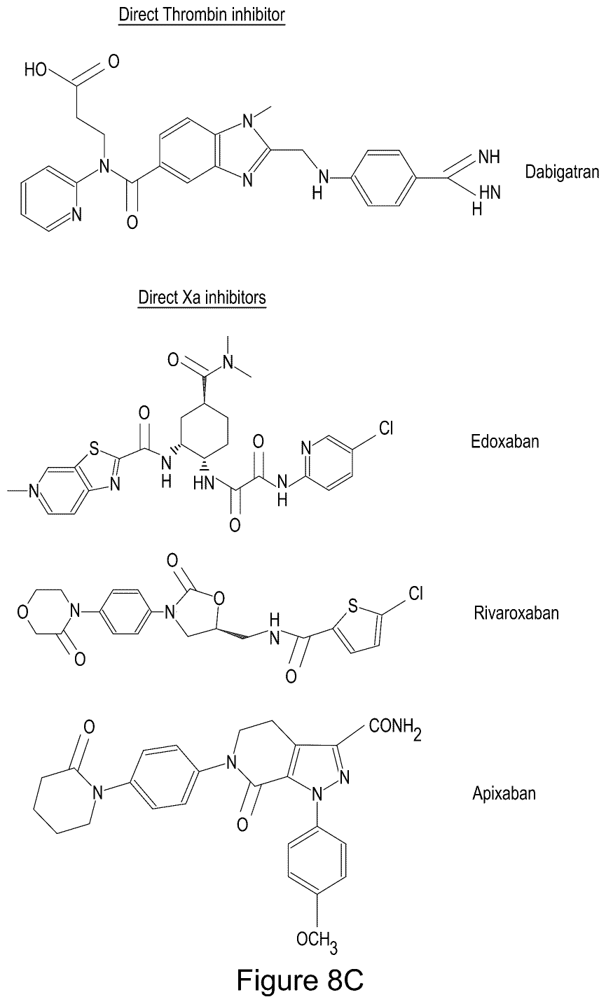

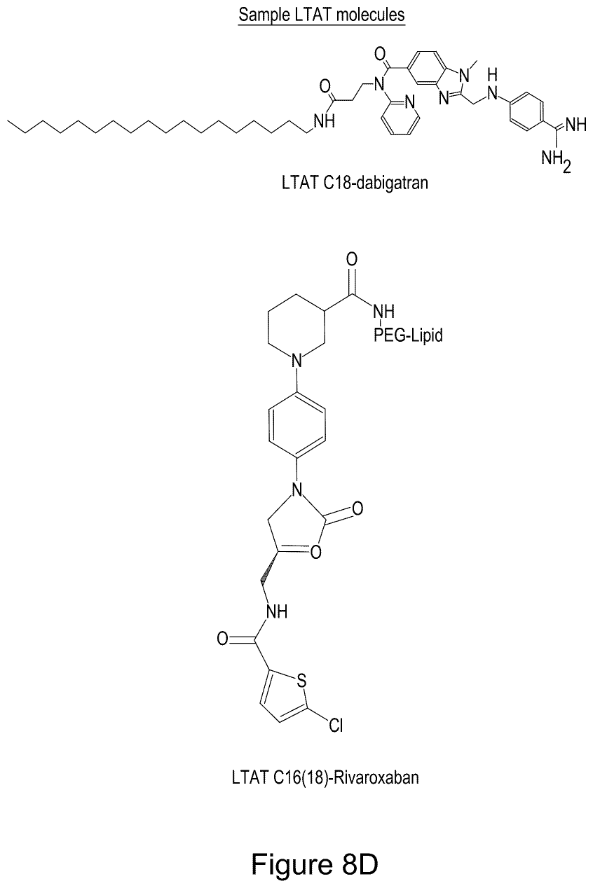

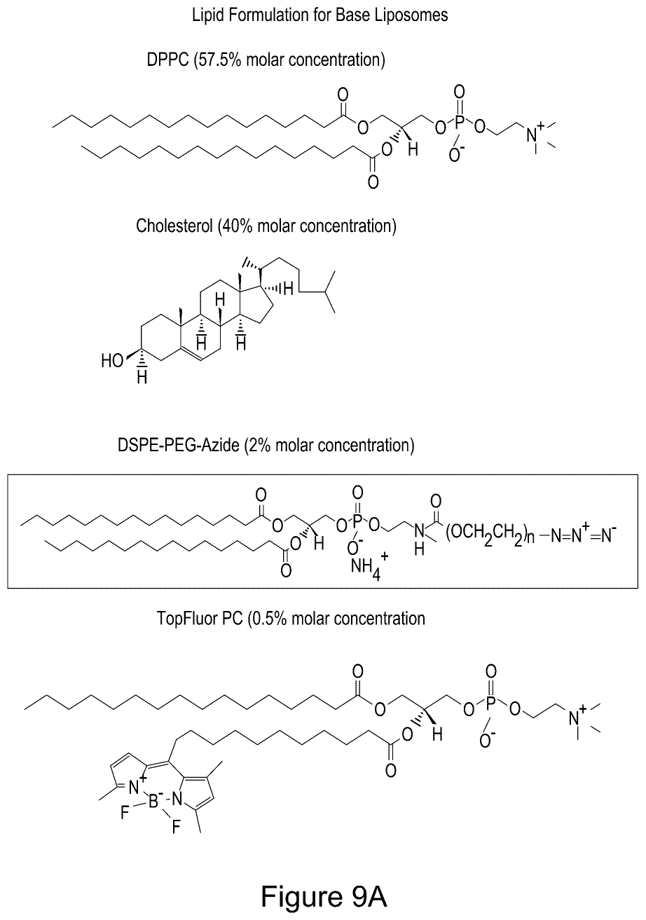

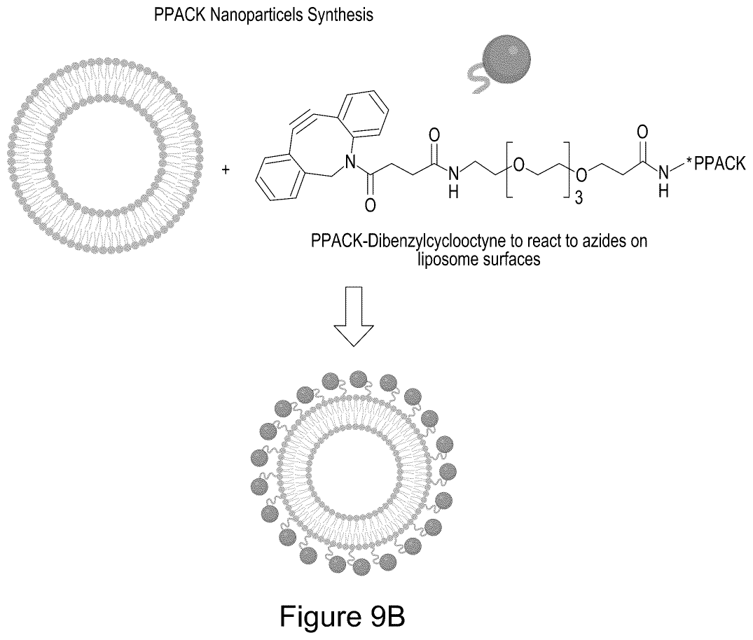

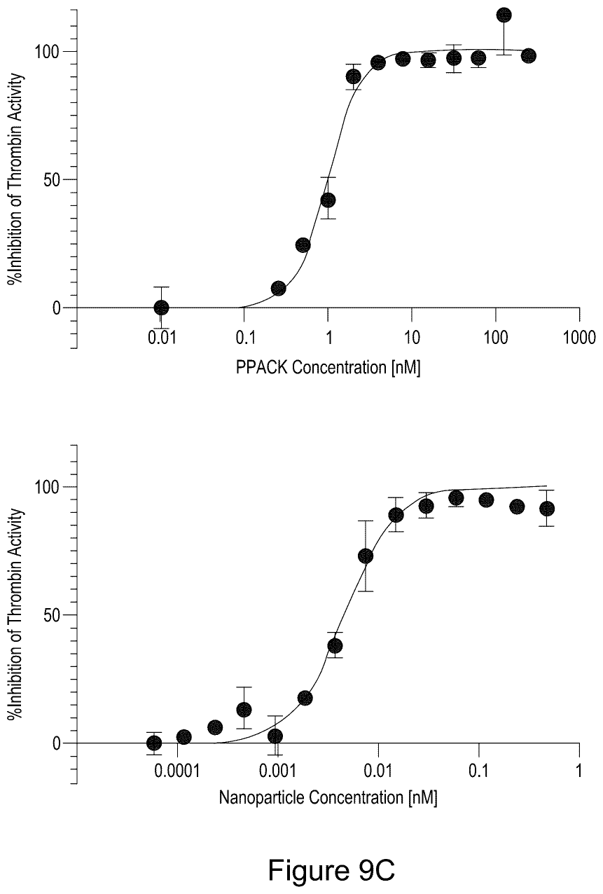

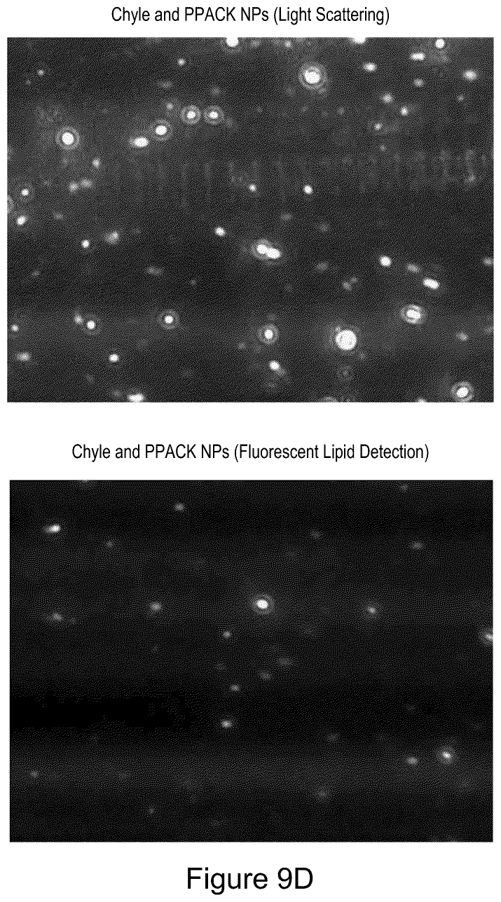

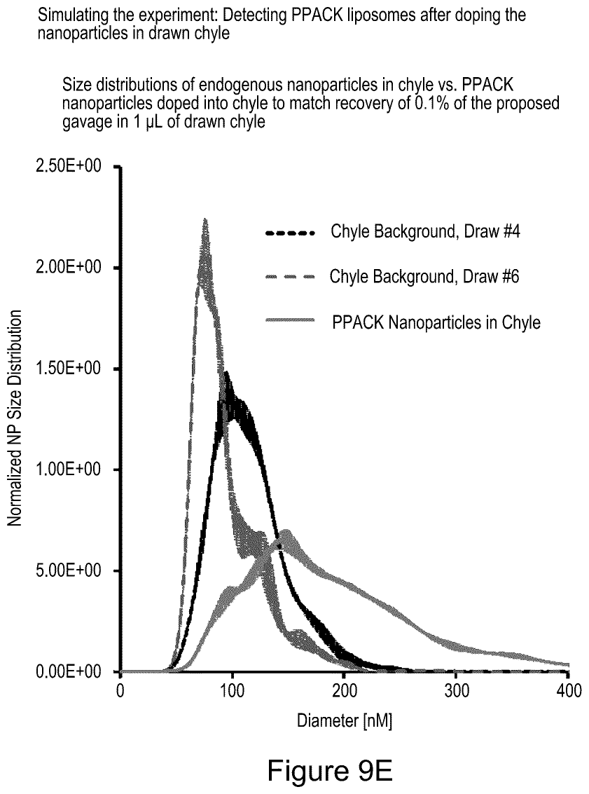

For the purpose of illustration, and not limitation, FIGS. 8A-9B schematically illustrate the process of creating lipid conjugate in accordance with the presently disclosed subject matter. A lipid formulation serves as the base for the attachment of the anticoagulant molecules, resulting in the formation of an anticoagulant-lipid nanoparticle (NPs).

The lipid conjugate of the presently disclosed subject matter can further include one or more additional components. For example, in certain embodiments, a spacer can be disposed between the various constituents of the lipid conjugates. Additionally, the spacer can reinforce the conjugation between the constituents of the lipid conjugate, if present, be used to control the relative positions of the lipids or lipid-like molecules with the anticoagulants. In certain embodiments, the spacer can comprise a polymer or a biomolecule. In certain non-limiting examples, the polymer can be polyethylene glycol, polyethylene, polyethylene glycol, dendrimers, polyacrylic acid, hydroxyethyl starch (HES), polylactide-co-glycolide, poly-D, L-p-dioxanonepoly lactic acid-ethylene glycol block copolymer (PLA-DX-PEG), poly (ortho) esters, poly-glutamate, polyaspartates, polymer of a-B-unsaturated monomers such as (meth) acrylic acid, crotonic acid, maleic acid, maleic anhydride, fumaric acid, itaconic acid/anhydride, etc., comonomers including vinyl ethers, vinyl esters, vinylamine amides, olefins, and/or diallyl dialkyl ammonium halides, preferably vinyl ether, poly (diethylenglycoladipate), polyethyleneimine, polyglycolide, polyurea, Polylimonen (=Polylimo), poly (2-methyl-1, 3-propyleneadipate), and graft polymers and graft (block) polymers, e.g., with other polymers. For example and not limitation, polyethylene glycol (PEG) n can be used as the spacer.

In certain non-limiting embodiments, the size and use of a spacer can be selected to control the overall size of the lipid conjugate. Thus, the size and use of a spacer can be based, at least in part, on the size of the bioconjugate. In particular embodiments, the size of the overall lipid conjugate can be controlled to induce the enhanced absorption and availability of the drug in the lymphatic system.

### 5.4 Methods of Treatment

In certain embodiments, the present disclosure provides for methods of treating an infectious, inflammatory or autoimmune disorder affecting the gut lymphatic system, comprising administering, to a subject in need of such treatment, an effective amount of LTAT molecule that prevent, reduces, or inhibits one or more sign or symptom of such disorder and without significantly altering blood coagulation kinetics in the subject.

In certain embodiments, the present disclosure provides for methods of treating or preventing lymphatic thrombosis associated with viral or bacterial infection in a subject comprising administering, to a subject in need of such treatment, an effective amount of a LTAT molecule for treating coagulation within the lymphatic system in the subject. In certain embodiments, the infection causes an inflammation which leads to lymphatic clot formation. In certain embodiments, the present disclosure provides a method of treatment and/or prevention of lymphatic thrombosis in a subject.

In certain embodiments, the present disclosure provides for methods comprising of treating lymphatic thrombosis associated with an inflammatory disorder in a subject including administering, to a subject in need of such treatment, an effective amount of a LTAT molecule for treating coagulation within the lymphatic system in the subject. In certain embodiments, the inflammatory condition is an inflammatory bowel disease, including but not limited to ulcerative colitis and Crohn's disease. In certain embodiments, the present disclosure provides a method of treatment and/or prevention of gut related inflammation resulting from lymphatic thrombosis lymphatic thrombosis.

In certain embodiments, the present disclosure provides for a method of treating or preventing one or more lymphatic clotting associated with viral or bacterial infection. In certain embodiments, the present disclosure provides for a method of treating or preventing lymphatic clotting associated with an inflammatory condition, including but not limited to inflammatory bowel diseases, such as ulcerative colitis and Crohn's disease.

### 5.5 Pharmaceutical Compositions

In certain embodiments, a pharmaceutical composition of the present disclosure includes a lipid conjugate, and a pharmaceutically acceptable carrier. Suitable carriers that can be used with the presently disclosed subject matter have the characteristics of not interfering with the effectiveness of the biological activity of the active ingredients, e.g., disclosed inhibitors/anti-cancer agents, and are not toxic to the patient. Non-limiting examples of suitable pharmaceutical carriers include phosphate-buffered saline solutions, water, emulsions, such as oil/water emulsions, various types of wetting agents, and sterile solutions. Additional non-limiting examples of pharmaceutically acceptable carriers include gels, bioabsorbable matrix materials, implantation elements containing the inhibitor and/or any other suitable vehicle, delivery or dispensing means or material. Such pharmaceutically acceptable carriers can be formulated by conventional methods and can be administered to the subject. In certain embodiments, the pharmaceutical acceptable carriers can include buffers such as phosphate, citrate, and other organic acids; antioxidants including ascorbic acid and methionine; preservatives (such as, but not limited to, octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol and m-cresol); low molecular weight (less than about 10 residues) polypeptides; proteins, such as serum albumin, gelatin or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). In certain embodiments, the suitable pharmaceutically acceptable carriers can include one or more of water, saline, phosphate buffered saline, dextrose, glycerol, ethanol or combinations thereof.

In certain non-limiting embodiments, the pharmaceutical compositions of the present disclosure can be formulated using pharmaceutically acceptable carriers well known in the art that are suitable for oral administration. Such carriers enable the pharmaceutical compositions to be formulated as tablets, pills, capsules, liquids, gels, syrups, slurries, suspensions and the like, for oral or nasal ingestion by a patient to be treated. In certain embodiments, the pharmaceutical composition is formulated as a capsule. In certain embodiments, the pharmaceutical composition can be a solid dosage form. In certain embodiments, the tablet can be an immediate release tablet. Alternatively or additionally, the tablet can be an extended or controlled release tablet. In certain embodiments, the solid dosage can include both an immediate release portion and an extended or controlled release portion.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the thromboembolic disorder.

Dosage forms (pharmaceutical compositions) suitable for administration may contain from about 1 milligram to about 1000 milligrams of active ingredient per dosage unit. In these pharmaceutical compositions the active ingredient will ordinarily be present in an amount of about 0.1-95% by weight based on the total weight of the composition

By way of general guidance, the daily oral dosage of each active ingredient, when used for the indicated effects, will range between about 0.001 to about 1000 mg/kg of body weight. Compounds of this invention may be administered in a single daily dose, or the total daily dosage may be administered in divided doses of two, three, or four times daily.

In certain embodiments, the present disclosure provides for pharmaceutical compositions including a LTAT molecule, as described herein, in a suitable pharmaceutical carrier. The amount of the LTAT molecule present in the composition can be calculated to provide, when administered to a subject in need of such treatment, an effective amount of the LTAT molecule.

In certain embodiments, the present disclosure provides for pharmaceutical compositions including therapeutically effective amounts of any of the LTAT molecule, for example but not limited to together with a pharmaceutical carrier such as water or other physiologic solvent. A therapeutically effective amount prevents, reduces or inhibits lymphatic clot formation.

In certain non-limiting embodiments, the LTAT molecule can be included in an oil solution, micelle, liposome, or a similar structure.

In certain embodiments, the pharmaceutical composition can be a liquid, including a LTAT molecule in a liquid pharmaceutical carrier including, for example, water (an aqueous carrier) or saline. In certain embodiments, said liquid composition can optionally further contain one or more of a buffer or a preservative.

In certain other embodiments, the pharmaceutical composition of the present disclosure can be a solid, for example in the form of a tablet, capsule, sachet or suppository, including a dose of a LTAT molecule that provides an effective amount of the LTAT molecule to a subject in need of such treatment when administered according to a dosing regimen. In certain embodiments, said solid pharmaceutical composition can further include one or more excipients, for example, but not limited to, lactose, sucrose, mannitol, erythritol, carboxymethylcellulose, microcrystalline cellulose, hydroxypropylmethylcellulose, starch, polyvinylpyrrolidone, etc.

In certain embodiments, a pharmaceutical composition can include an additional agent that has antimicrobial and/or anti-inflammatory activity. In certain embodiments, such compound includes, but is not limited to, an antibiotic agent, a steroid, or a non-steroidal anti-inflammatory agent. In certain other embodiments, the pharmaceutical composition can include an analgesic agent. In certain further embodiments, the pharmaceutical composition can include an agent that dissolves pre-existing lymphatic clots such as tissue plasminogen activator (tPA) or activated plasminogen.

Pharmaceutically acceptable salts are art-recognized, and include relatively non-toxic, inorganic and organic acid addition salts of compositions of the disclosed subject matter, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di-, or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenthylamine; (trihydroxymethyl) aminoethane; and the like, see, for example, J. Pharm. Sci., 66:1-19 (1977).

### 5.6 Kits

The presently disclosed subject matter further provides kits containing materials useful for performing the methods and compositions disclosed herein. For example, but not by way of limitation, any combination of the materials useful in the present disclosure can be packaged together as a kit for performing any of the disclosed methods or use of the compositions.

In certain embodiments, a kit of the present disclosure can contain the LTAT molecules together with a suitable pharmaceutical carrier. In certain embodiments, the kit components can be packaged in single use form, suitable for carrying one a single administration.

In certain embodiments, the kit further includes a package insert that provides instructions for using the components provided in the kit. For example, a kit of the present disclosure can include a package insert that provides instructions for using LTAT molecules provided in the kit.

Alternatively or additionally, the kit can include other materials desirable from a commercial and user standpoint, including other buffers, diluents and filters. In certain embodiments, the kit can include materials for preparing LTAT molecules.

Kits can supply reagents in pre-measured amounts so as to simplify the performance of the subject methods and administration of the composition. Optionally, kits of the present disclosure include instructions for performing the method or administering the composition. Other optional elements of a kit of the present disclosure include suitable buffers, reagents, packaging materials, etc. The kits of the present disclosure can further include additional reagents that are necessary for performing the disclosed methods and use of compositions. The reagents of the kit can be in containers in which they are stable, e.g., in lyophilized form or as stabilized liquids.

### 6. EXAMPLE

The presently disclosed subject matter will be better understood by reference to the following Example, which is provided as exemplary of the presently disclosed subject matter, and not by way of limitation.

### Results

### Antithrombotic Properties of the Lymphatic Endothelium.

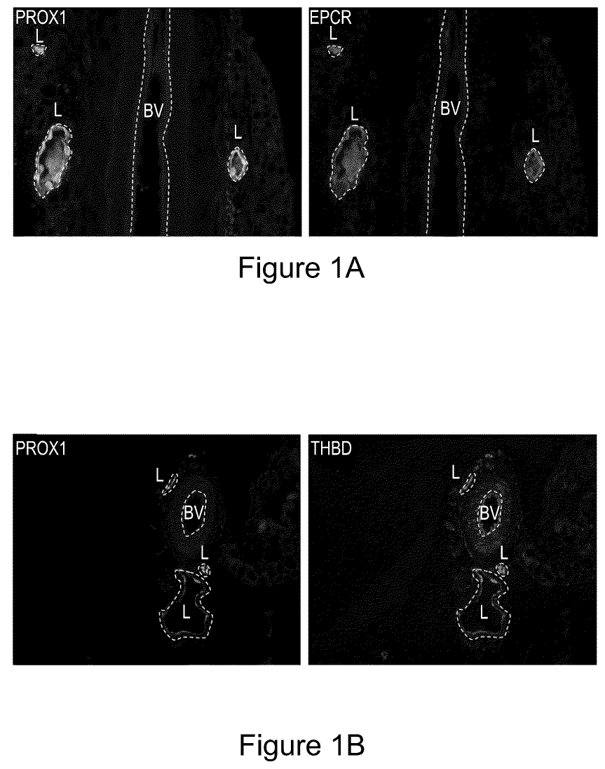

The present disclosure showed that the lymphatic endothelium that lines the lymphatic vessels expressed a family of transcription factors, including FOXC2, PROX1, and GATA2, which play a key role in the development of the lymphatic system, in particular the lymphatic valves, and are also associated with a potent antithrombotic phenotype. The antithrombotic phenotype was observed also in venous valve endothelium, indicated by the expression FOXC2 and PROX1, and plays a role in preventing blood clots from forming within the pocket of venous valves. The antithrombotic phenotype included the down regulation of several prothrombotic and proinflammatory endothelial surface proteins including: von Willebrand Factor and P-selectin, and the upregulation of antithrombotic endothelial surface proteins thrombomodulin (THBD) and endothelial protein C receptor (EPCR) (FIGS. 1A and 1B). The lymphatic endothelium, especially the lymphatic network in the gut, shared this anti-thrombotic expression pattern marked by high THBD and EPCR expression. The lymphatic endothelium showed a synergistic effect, such that prothrombotic proteins were reduced and antithrombotic proteins were increased, which in turn produced an additive response and created a strong inhibition of clot formation.

### PAR1-Tango Mouse Model Reports Thrombin Activity In Vivo.

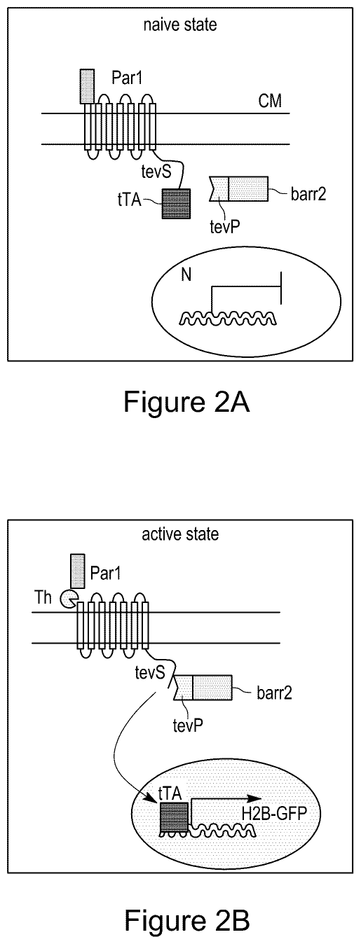

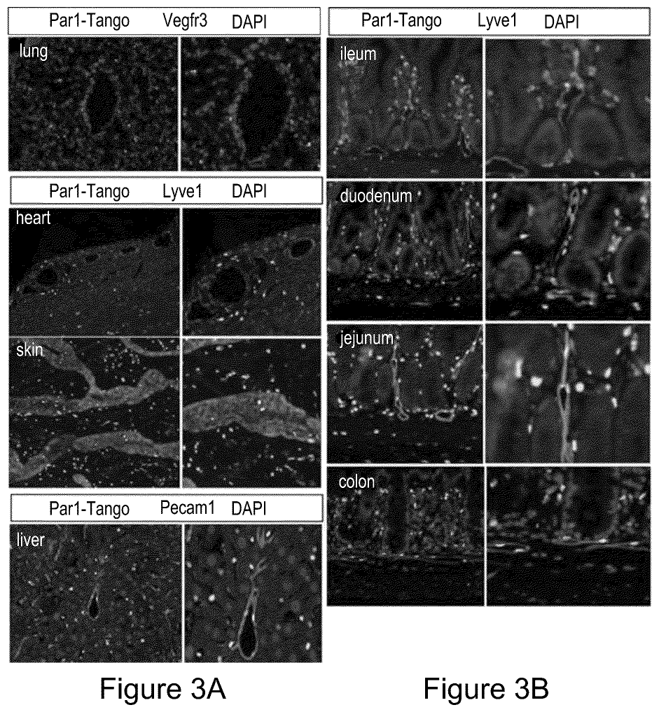

The present disclosures provides a mouse model, harboring a PAR1-Tango allele, as a reporter of thrombin expression, the main procoagulant factor, under normal and healthy conditions (FIGS. 2A and 2B). Mice expressing the PAR1-Tango allele showed strong thrombin activity in the gut lymphatics (FIG. 3B), whereas the lack of reporter activity, when the PAR1 receptor is not exposed to thrombin, was observed in the lung, heart, skin, and liver (FIG. 3A). The present disclosure shows the gut lymphatic environment is naturally procoagulant.

### Lymphatic Clot Formation in Response to Gastrointestinal Infection.

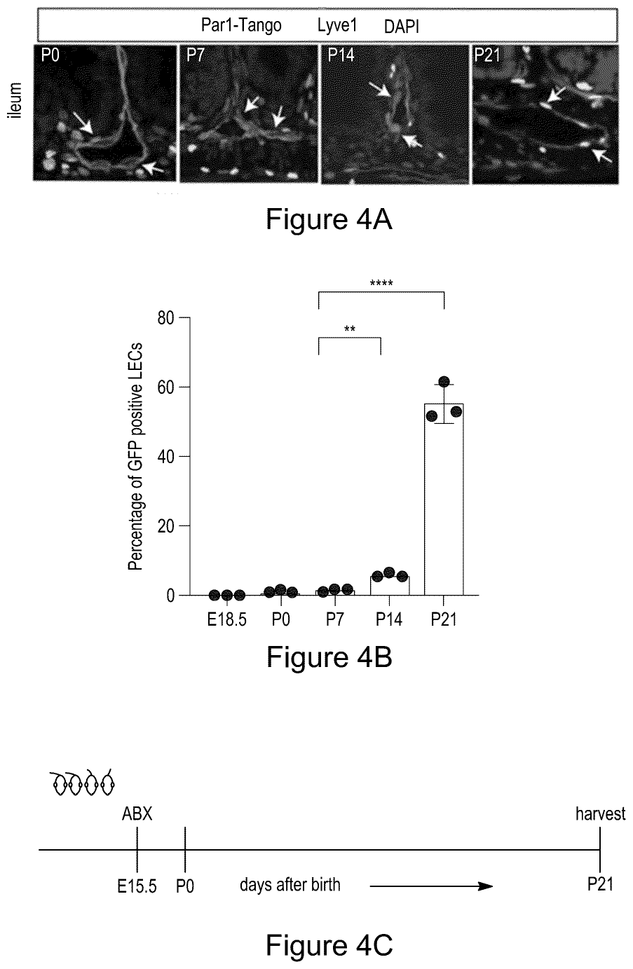

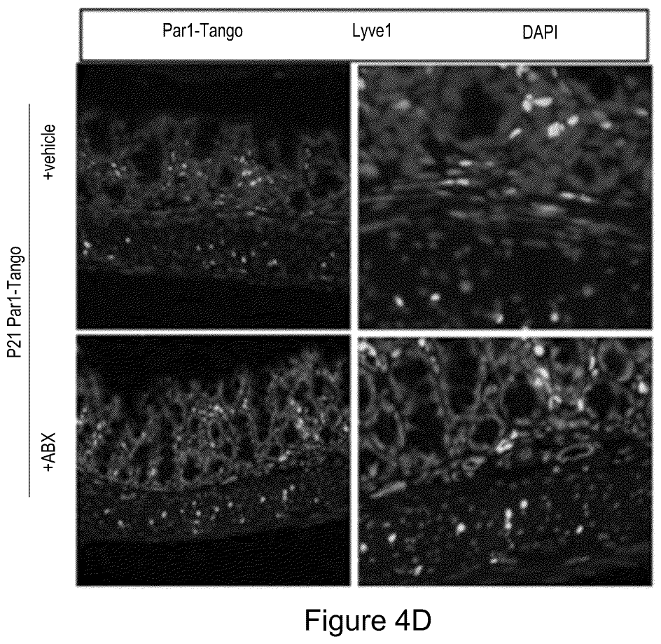

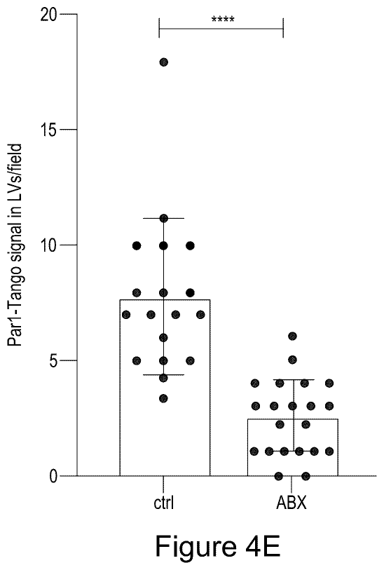

The present disclosure showed that lymphatic fibrin clot formation is a critical part of the gut's natural immune response working to prevent the spread of pathogenic bacteria introduced into the gut of mice (FIGS. 4A-4E). As shown in FIGS. 4A and 4B, PAR1-Tango activity arises in coordination with the gut microbiome. Lymphatic vessels (LYVE1 positive) were found to be negative for PAR1-Tango expression at P0 and P7 but showed expression (GFP-positive cells) at P14 and had even stronger expression at P21. The present disclosure demonstrated that the PAR1-Tango positive cells are present in the neonatal intestine.

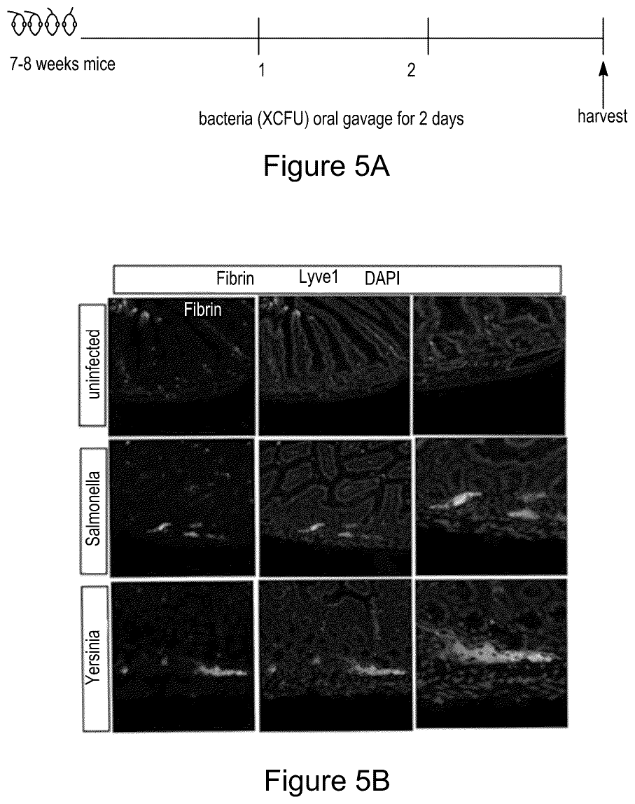

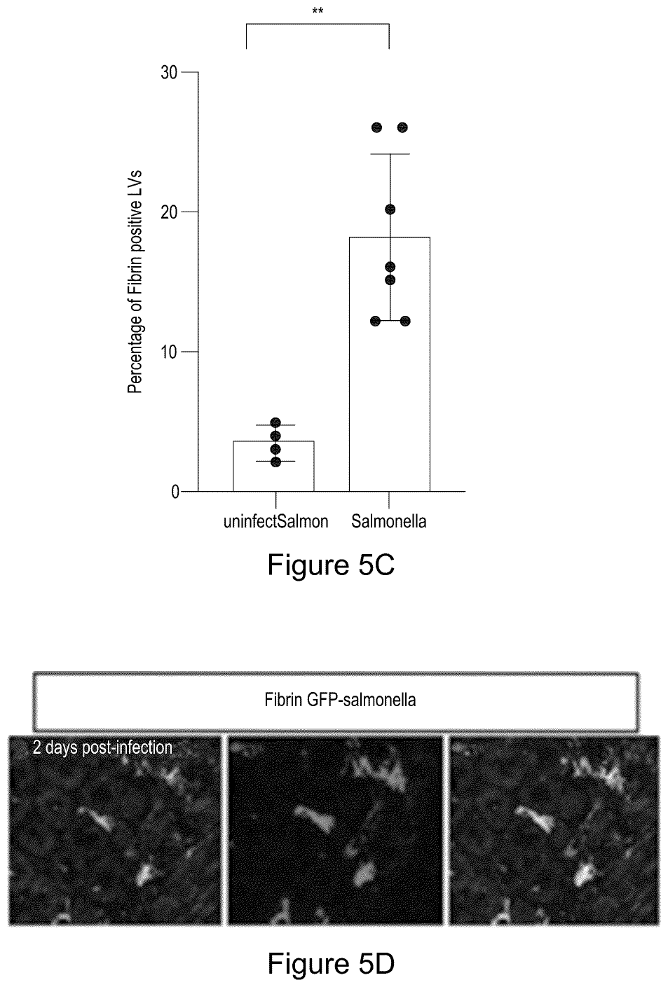

Clot formation within the gut lymphatic vessels can block the flow of fluid, immune cells, and bacteria preventing dissemination of the infection and allowing more time for the activated immune cells to respond to the invading bacteria and impeding spread of the pathogen from the gut to other sites in the body (i.e. sepsis). Similarly, in viral infections gut lymphatic clot formation occurs and where it functions to prevent pathogen dissemination and concentrates local activated immune cells to the area to eliminate pathogens. In both of these cases lymphatic clotting is beneficial to health. The present disclosure shows that the inhibition effect of neonatal antibiotic (“ABX”) treatment on PAR1-Tango reporter activity, confirming that thrombin generation is linked to bacterial colonization of the gut. The present disclosure established a connection between the bacteria in the gut lumen to thrombin activity in the gut lymphatic lumen. The present disclosure further demonstrates that bacterial infections created lymphatic clots. As shown in FIGS. 5A-5D, mature mice exposed to Salmonella or Shigella orally through gavage form fibrin clots in LYVE1-positive lymphatic vessels. The present disclosure shows that approximately 20% of gut lymphatic vessels were thrombosed after Salmonella infection (FIG. 5C). This was further demonstrated in mice gavaged with GFP-expressing Salmonella bacteria, which showed GFP-expressing Salmonella found at sites of fibrin clot formation within the gut lymphatic vessels (FIG. 5D).

### Gut Inflammation Leads to Gut Lymphatic Clot Formation.

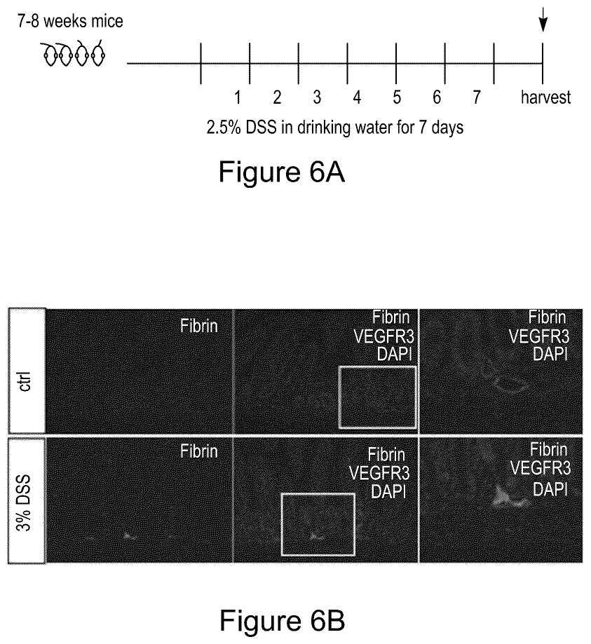

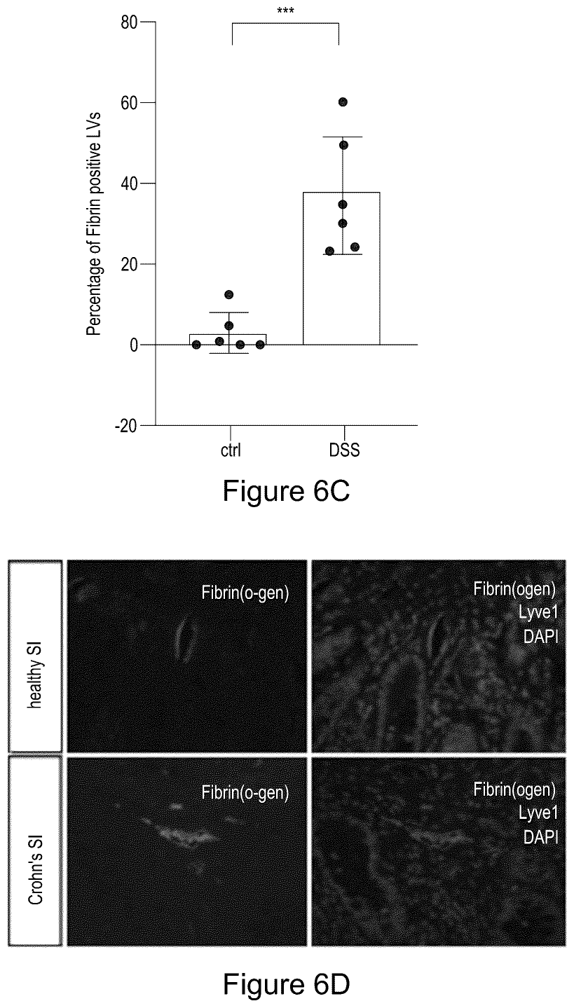

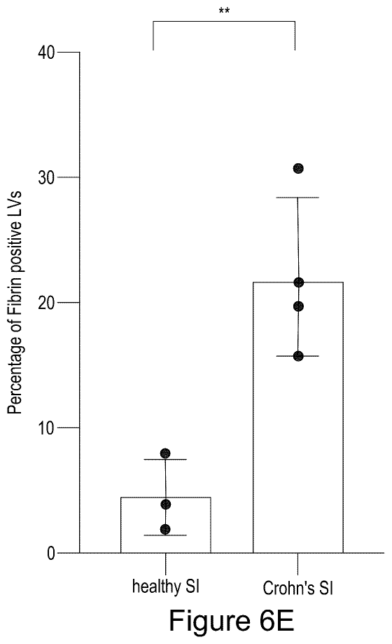

The present disclosure found that the induction of lymphatic clots can occur inappropriately in the setting of inflammation caused by non-pathogenic sources, and autoimmune or inflammatory disease. In these cases the activated immune cells, that are known to be thrombotic, may be activating intralumenal thrombin activity in the lymphatic vessels, thereby triggering fibrin clot formation. Mouse models of bowel inflammation (induced with dietary ingestion of DSS) and human samples from chronic inflammatory bowel disease patients both show significant amounts of lymphatic clot formation relative to healthy controls (FIGS. 6A-6E). In these instances, the clots are not preventing the dissemination of a pathogen but are preventing the clearance of activated immune cells, thereby exacerbating inflammation and contributing to tissue damage and symptoms. Clinical data from patients with chronic inflammation of their gut show that they can develop nutrient deficiencies consistent with dysregulation of dietary lipid uptake, consistent with disrupted gut lymphatic flow.

### Native Anticoagulant Molecules Expressed by Gut Lymphatic Vessels Prevent Gut Lymphatic Clotting.

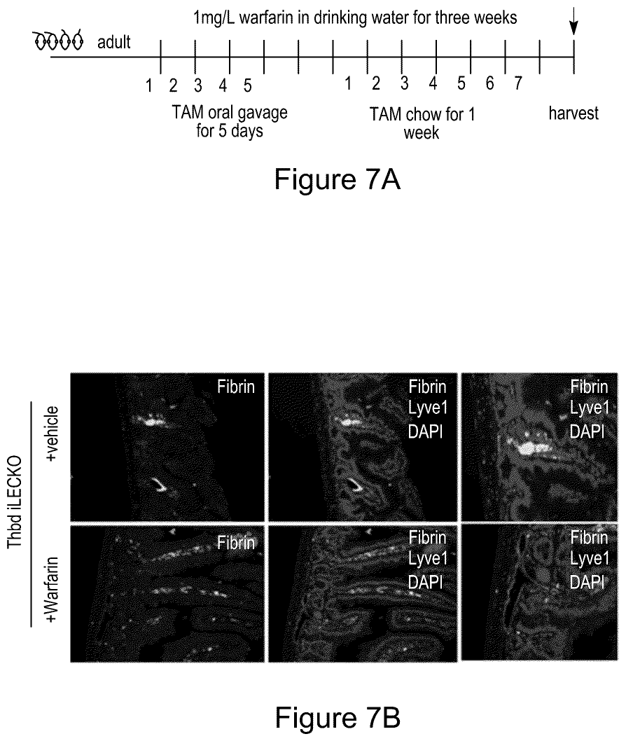

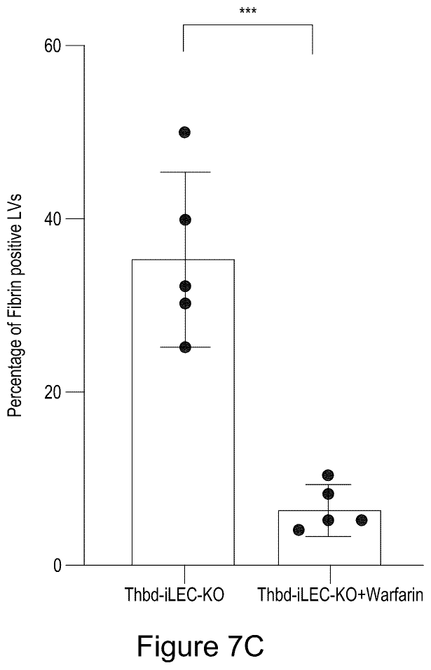

The present disclosure demonstrated that in mice genetic deletion of lymphatic endothelial THBD resulted in widespread gut lymphatic clotting and impeded gut lymph flow. Oral administration of the anticoagulant warfarin prevented lymphatic clotting in this context (FIGS. 7A-7C). This demonstrates that antithrombotic drugs can prevent lymphatic clot formation and lead to the restoration of lymphatic function. However, dosing with existing antithrombotic drugs will confer systemic anticoagulation, with established significant risks of bleeding throughout the body, including in the gut and brain. The risks of systemic anticoagulation, i.e. anticoagulation that affects both the blood and lymphatic vascular systems, are significantly higher in patients with chronic inflammation as they have higher rates of clinical bleeding, particularly in the gut. Therefore, there is a clear need for a functional antithrombotic drug that can be delivered and act preferentially to the gut lymphatics at a therapeutic dose. In addition, the volume of lymphatic fluid in the gut is much less than the volume of blood in the body, which means that a therapeutic dose of drug directly to the gut lymphatics will be massively diluted once it reaches the blood stream to well below therapeutic levels, thereby preventing systemic anticoagulation and inappropriate bleeding.

### Lipid Conjugates for Targeting Antithrombotic Drugs to the Gut Lymphatics.

The present disclosure demonstrated that lipid conjugate molecules with various lengths of flexible linkers can be connected to orally-active anti-thrombin molecules for the purpose of specific lymphatic-targeted antithrombotic drug delivery. The attachment of the antithrombotic drug to the head groups of lipids creates various LTAT molecules (FIGS. 8A-8D). Such molecules can be formulated as isolated lipids, as micelles, or in lipidic nanoparticles, including liposomes of solid lipid nanoparticles. Such molecules will be used in clinical cases of small bowel inflammation (such as Crohn's Disease) to reduce intensity and duration of inflammation of the bowel, and for any viral or bacterial infections where lymphatic clotting might be impeding the access of drugs to the cells within the clots. The molecules can be delivered in oil capsules orally and the molecules will be taken up specifically into the gut lymphatics as chylomicrons with the antithrombotic drugs exposed to the intralumenal lymphatic fluid milieu, where it will be able to block thrombin or Xa enzymatic activity that is required for clot formation. The LTAT molecules can be delivered as free lipids within an oil solution, or as micelles, or liposomes to enhance absorption and availability of the drug in the lymphatic system (FIGS. 8A and 8B). The LTAT molecules can be the only lipids used to form the micelles or liposomes or could be prominent components of the micelles or liposomes. Unabsorbed drug would be passed through the gut more readily than a non-lipid based small molecule and will not be readily absorbed into the blood stream, which can reduce the chance of the blood level of anti-thrombotic drug from becoming so high as to cause bleeding.

The process of creating LTAT molecules is illustrated in FIGS. 9A and 9B. Initially, a base liposome was created using a DSPE-PEG-azide formulation at a 2% molar concentration. This formulation ensured that the azide group was positioned on the outer surface of the liposome, facilitating the attachment of the anticoagulant molecules. After the liposome formation, PPACK-dibenzocyclooctyne, an exceptionally potent and selective irreversible inhibitor of thrombin, was attached to the outer surface of the liposome. The resulting PPACK nanoparticle (NP) retained anti-thrombotic activity, as demonstrated by a chromozym TH assay. PPACK NPs exhibited an IC50 value of approximately 0.8 nM, whereas the standalone PPACK had an IC50 value of around 0.0002 nM. Hence, liposomes proved to be highly effective for loading PPACK, with an estimated count of approximately 400 PPACK molecules per liposome (FIG. 9C). To mimic the effects of oral administration and investigate changes in nanoparticle size, PPACK NPs were incorporated into mouse-derived chyle (FIGS. 9D and 9E).

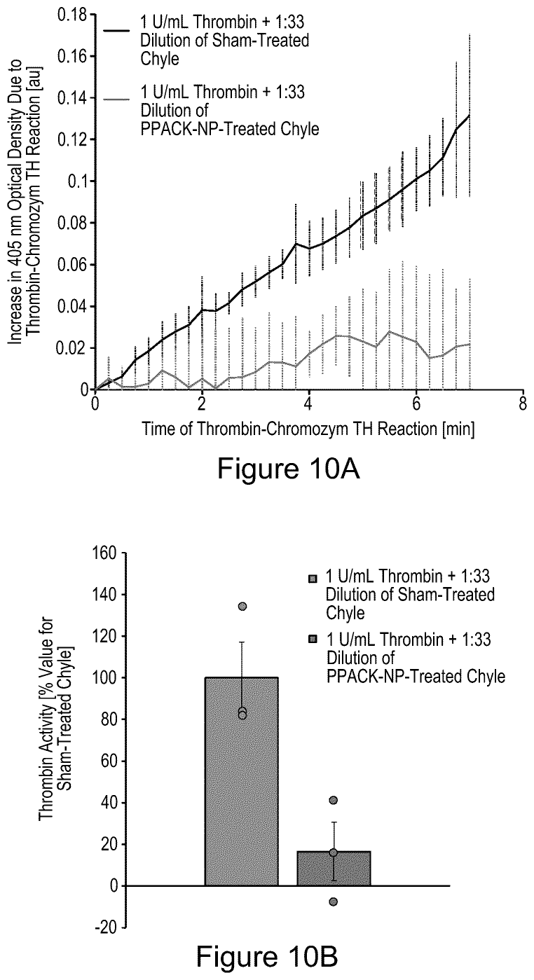

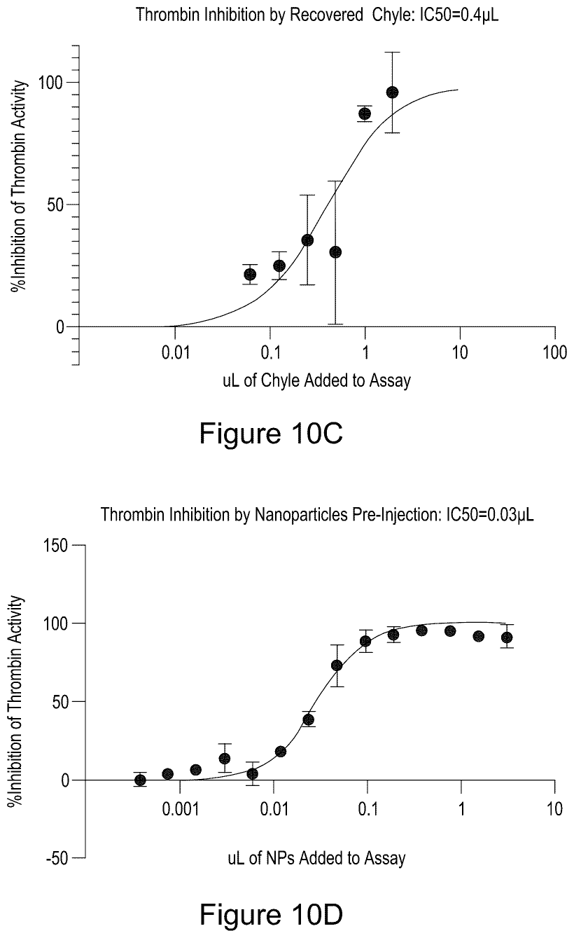

As demonstrated in FIGS. 10A and 10B, oral administration of PPACK NPs resulted in thrombin inhibition in chyle. Thrombin activity was assessed for chyle diluted 1:33 in 1 U/mL thrombin. Dilution of chyle from mice treated with oral PPACK NPs demonstrated a 1 U/mL thrombin inhibition IC50 at 0.4 μL chyle (FIG. 10C), while pre-injected PPACK NPs had an IC50 at 0.03 μL (FIG. 10D). Data indicates that each μL chyle was found to contain the equivalent of approximately 0.075 μL (0.03/0.4) of PPACK NP at their original concentration. An initial dose containing 250 μL of PPACK NPs (condensed to 50 μL and added to 400 μL of olive oil) produced one μL of gavage containing the equivalent of 0.56 μL of chyle (250/450). This equated to 13.4% of the gavage dose activity in the chyle at the time of recovery.

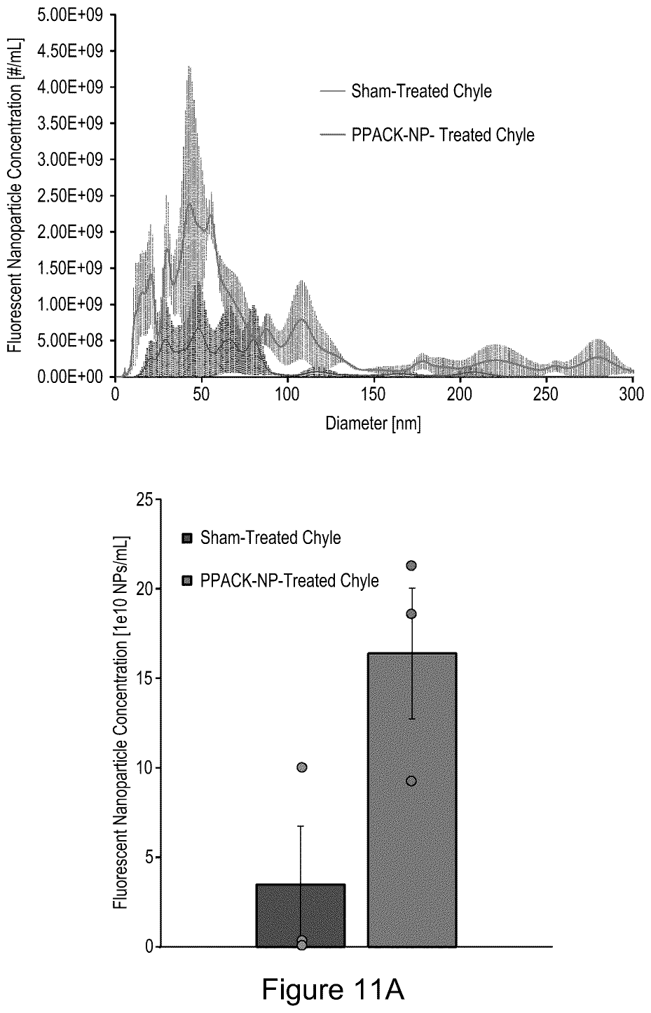

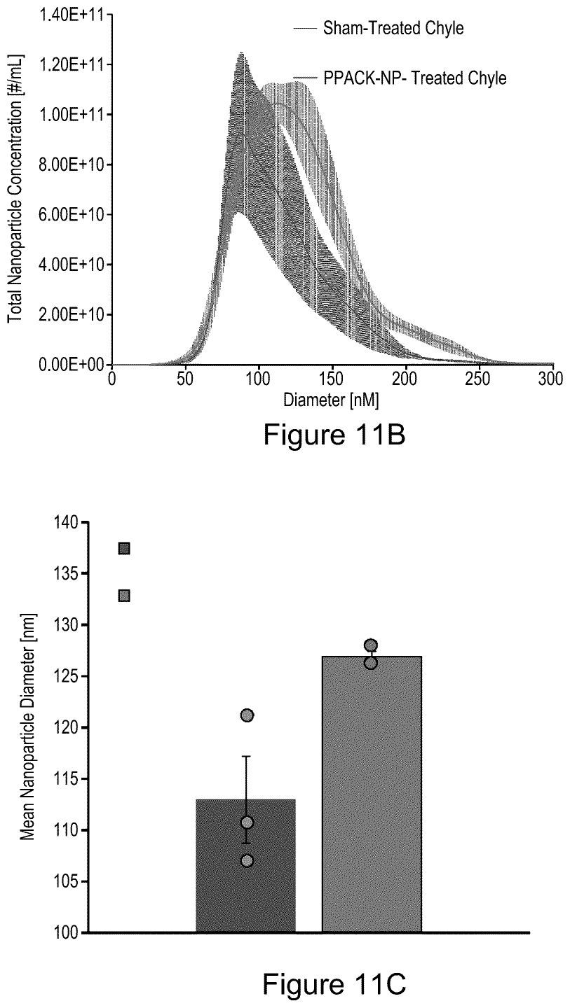

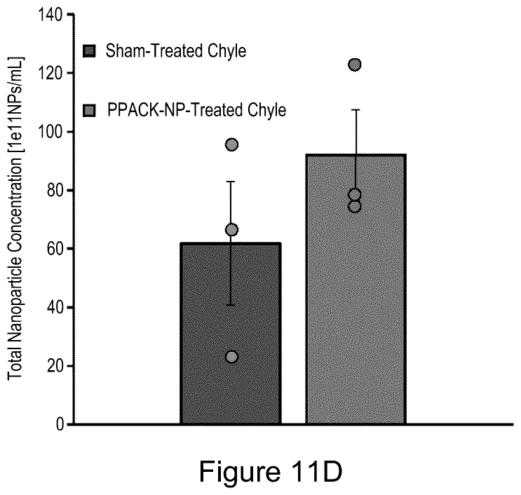

Fluorescence nanoparticle tracking analysis (F-NTA) was employed to evaluate the transport from oral gavage to chyle of fluorescent lipids formulated in the PPACK NPs (FIG. 11A). In chyle samples from mice treated with oral PPACK NPs, F-NTA determined the size and concentration of nanoparticles containing fluorescent lipids from the PPACK NPs. The present example shows that the size of nanoparticles within chyle containing fluorescent lipids did not correspond to that of the pre-injected nanoparticles, demonstrating that lipids from the PPACK NPs were incorporated in chylomicrons/endogenous vesicles, rather than being transported as part of intact nanoparticles. Results indicated a shift in size of the nanoscale lipid vesicles/aggregates found in chyle of mice treated with oral PPACK NPs, compared to the sham-treated mice, as demonstrated in FIG. 11B-11D.

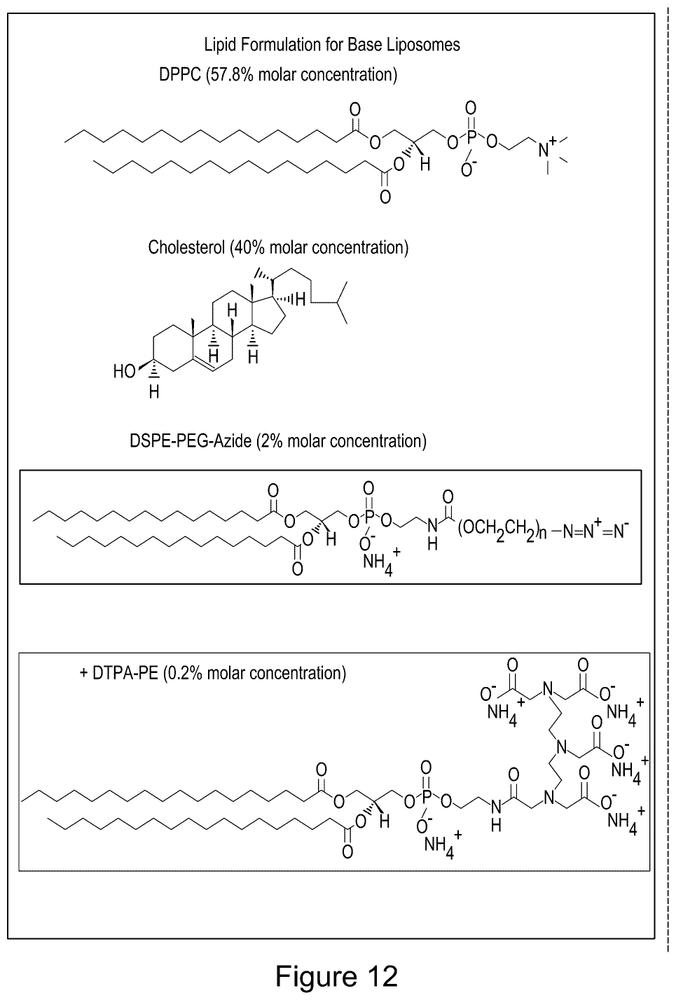

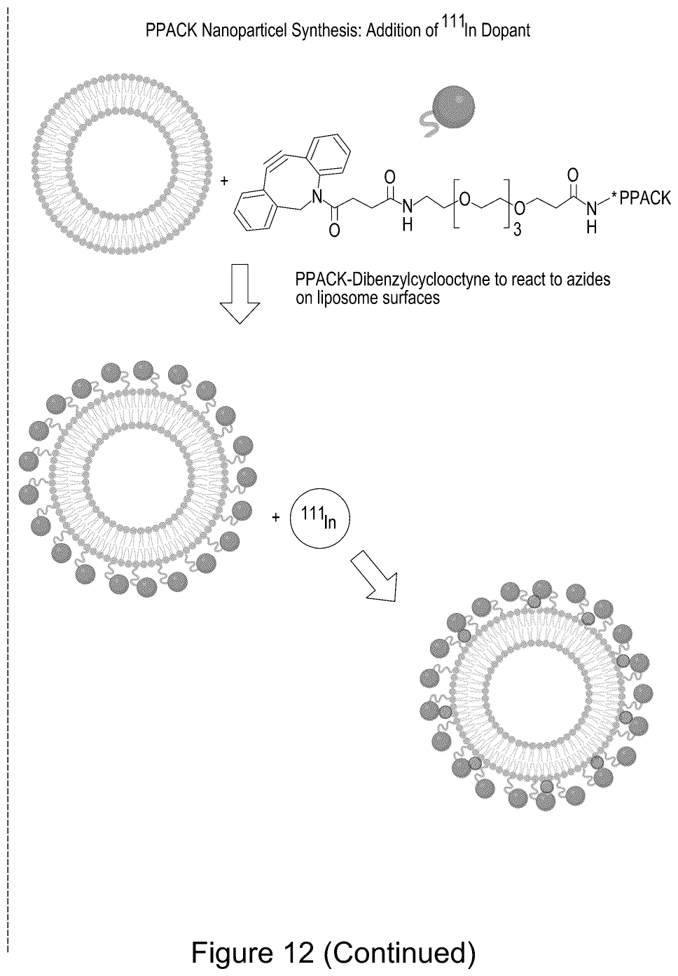

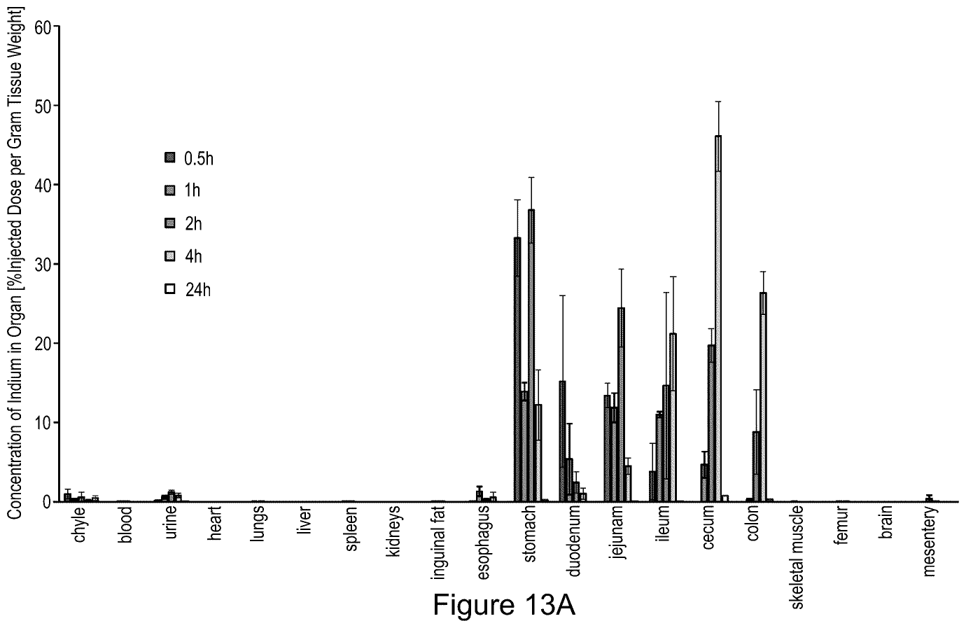

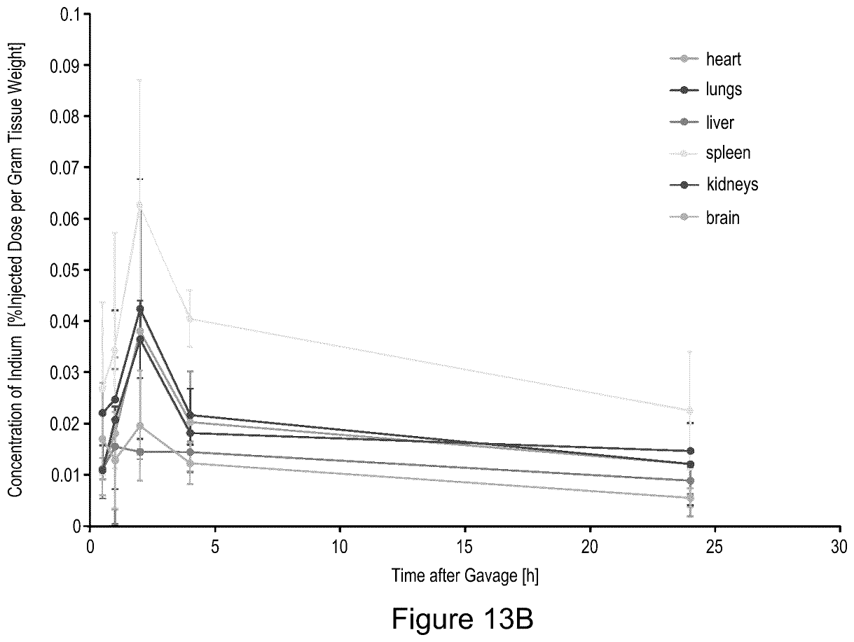

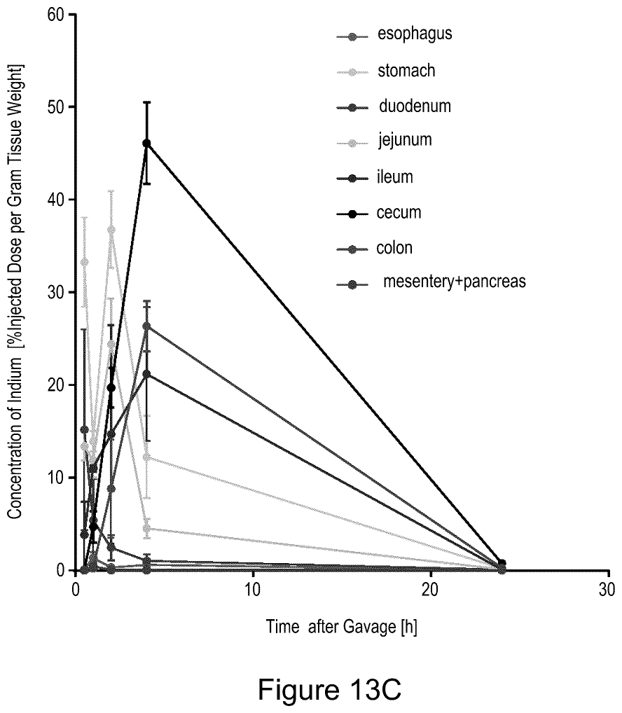

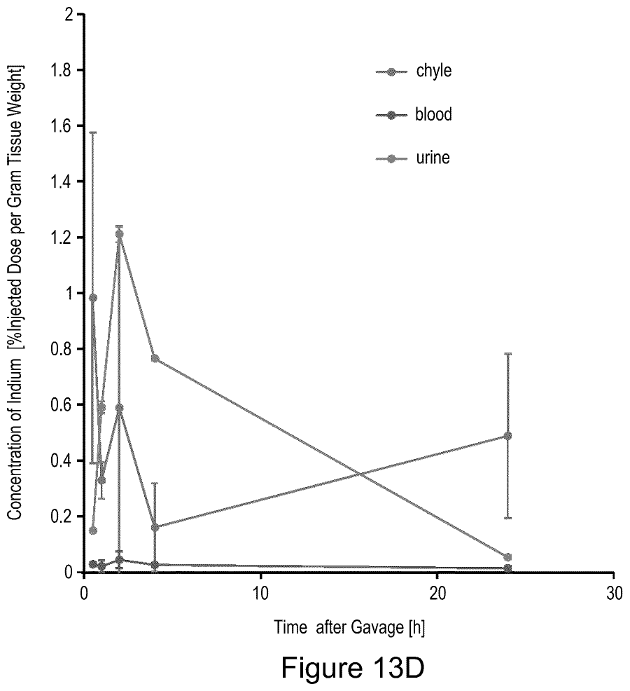

In order to assess the distribution of PPACK NPs in the body, a radiolabeling agent (111)In was incorporated into the NPs (as shown in FIG. 12). The movement of the radiolabeled tracer along the gastrointestinal tract of mice was tracked for a period of 24 hours after administration. The results revealed that the tracer molecule accumulated in various parts of the gastrointestinal tract, including the stomach, duodenum, jejunum, ileum, cecum, colon, and pancreas mesentery. However, after 24 hours (FIGS. 13A and 13C), the tracer had been cleared from these tissues. The tracer was found to have permeated multiple organs, such as the heart, lungs, liver, spleen, kidneys, and brain, at much lower concentrations than in tissues of the gastrointestinal tract. The levels of the tracer in all tissues gradually decreased and returned to normal within 24 hours (FIG. 13B). Only slight amounts of the tracer were detected in the blood, and urine analysis revealed its excretion at the 24-hour mark, while detection of the tracer persisted in the chyle.

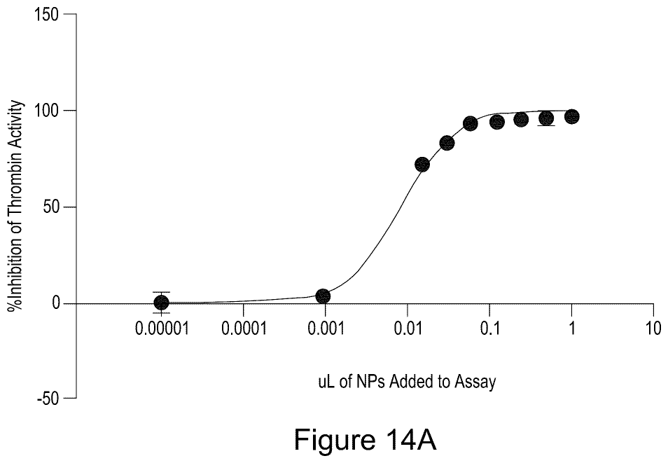

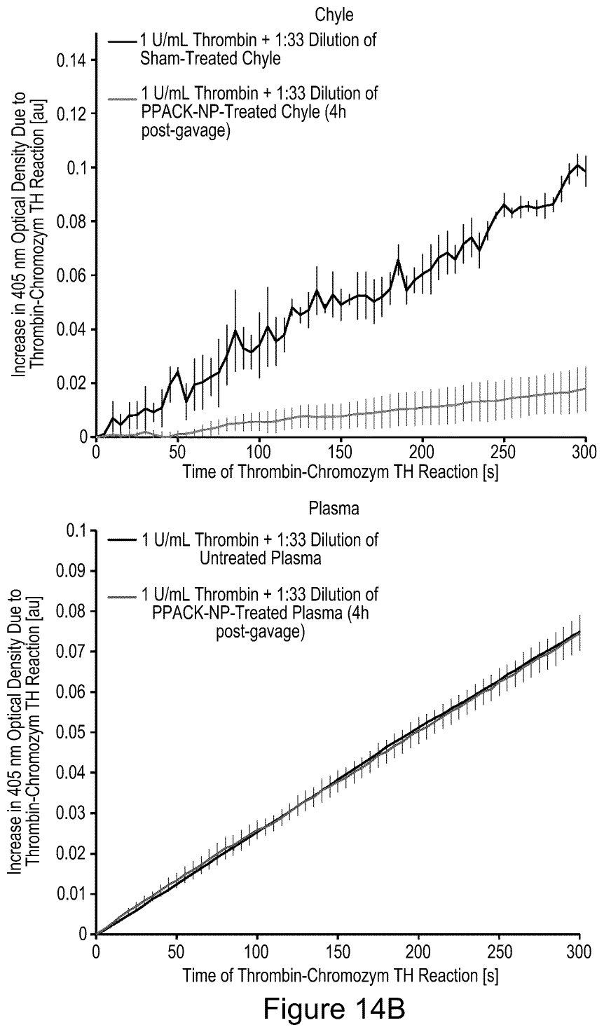

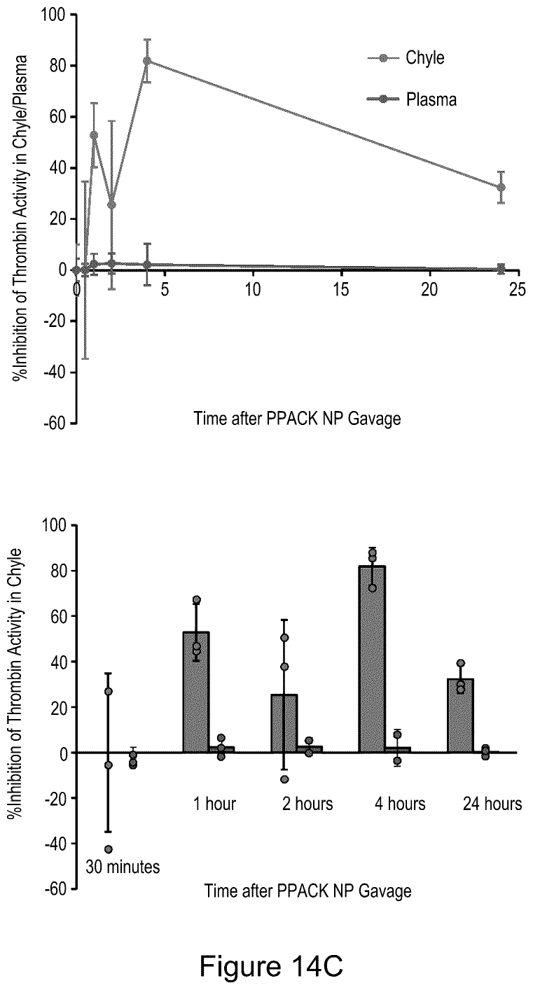

It was found that thrombin in plasma was not inhibited by treatment with oral gavage of PPACK NPs. FIG. 14A shows thrombin inhibition by PPACK-NPs prior to gavage (0.0085 μL IC50). FIG. 14B demonstrates that thrombin was inhibited in chyle, but not plasma, from mice treated with PPACK NP oral gavage. Thrombin inhibition in chyle increased in a noisy time course from 0-4 hours post-gavage, and significant inhibitory effects were demonstrated at 24 hours post-gavage (FIG. 14C).

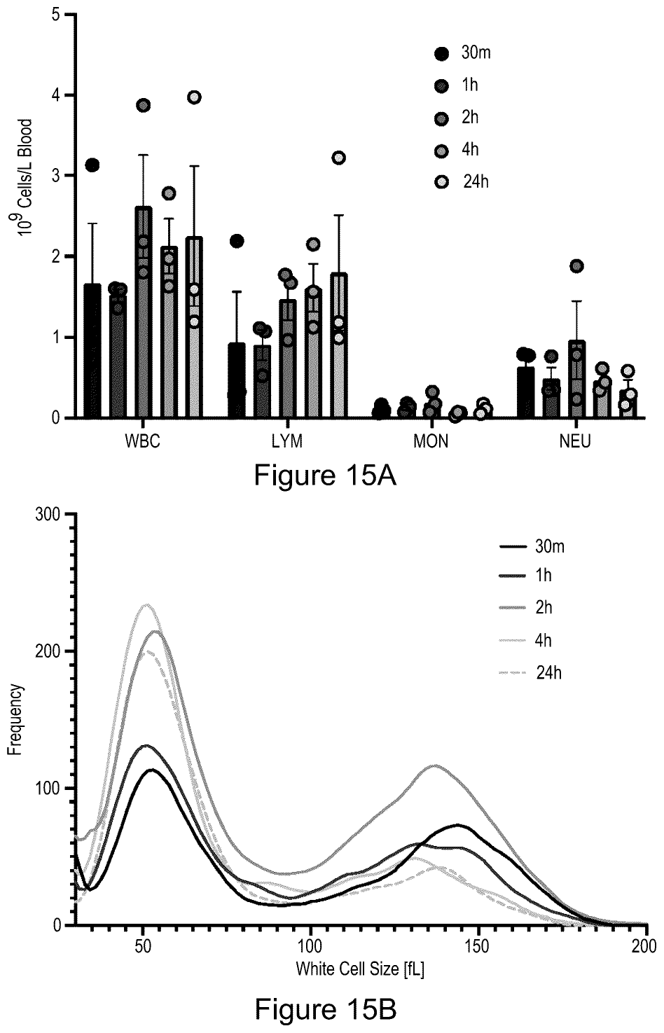

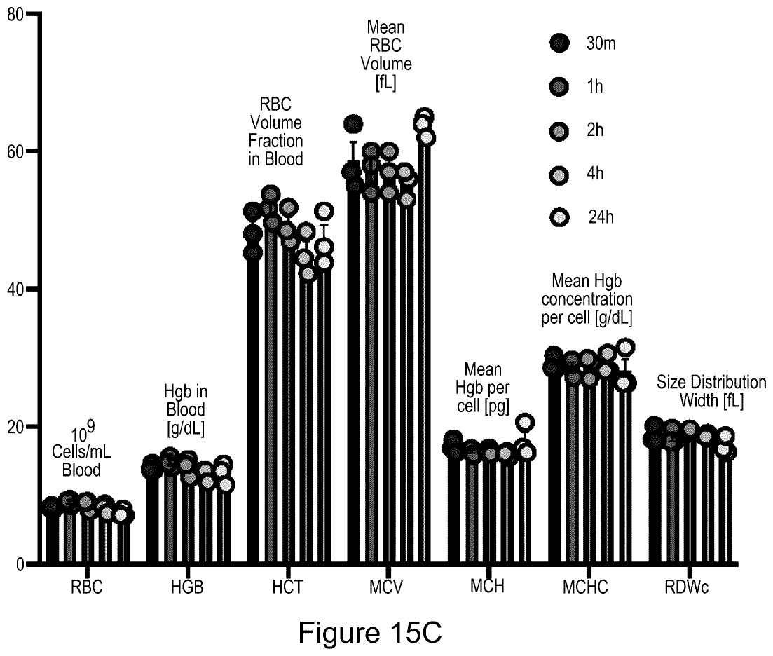

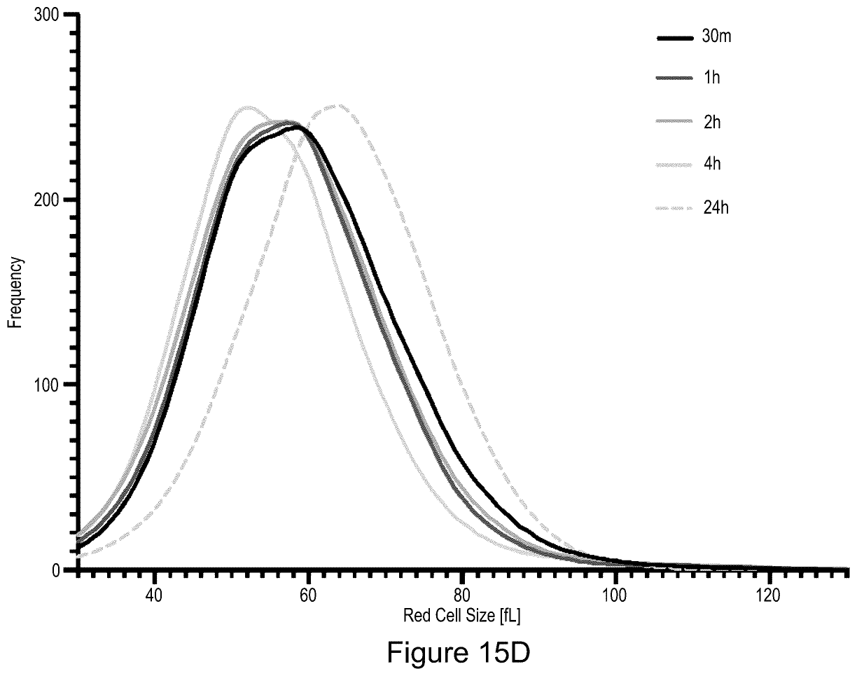

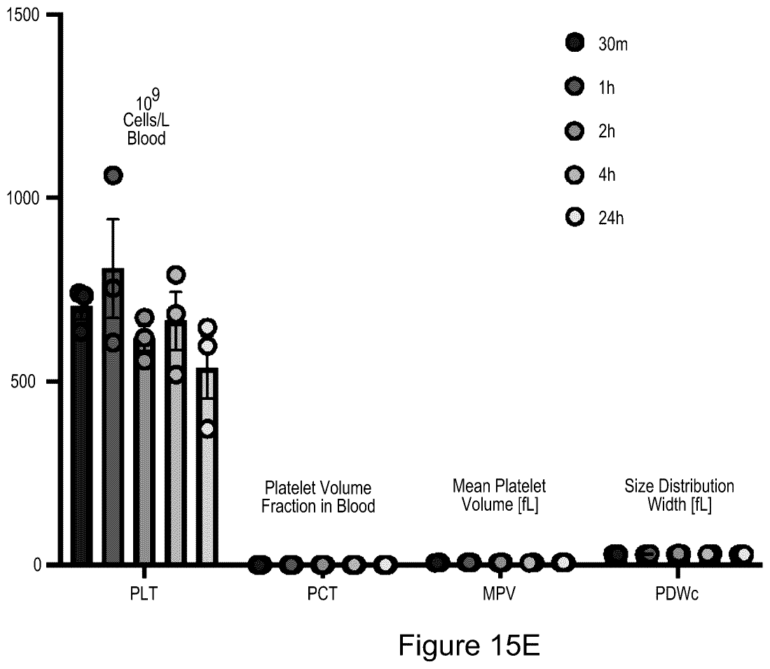

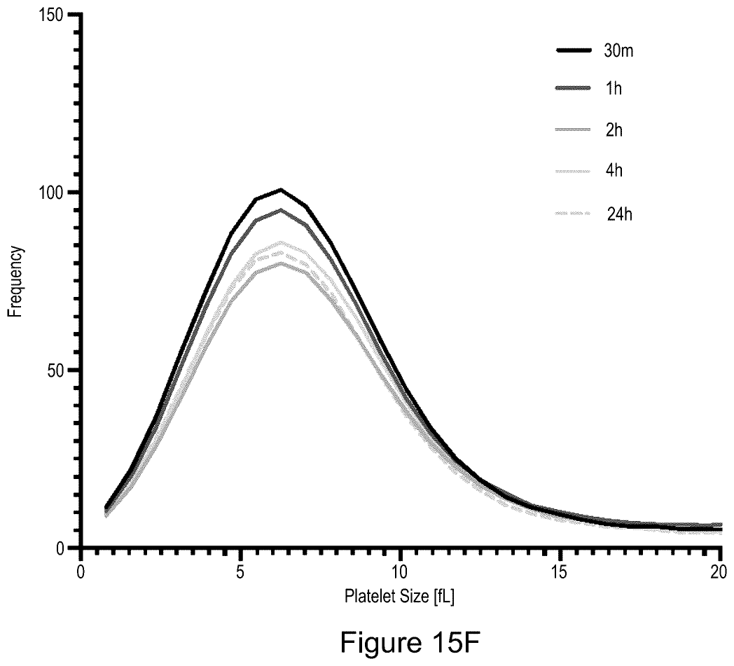

Blood tests were conducted at various time points within a 24-hour period following the gavage of the PPACK NPs. An analysis of the complete blood count, including white blood cells (WBC), lymphocytes (LYM), monocytes (MON), and neutrophils (NEU) demonstrated mild lymphopenia at early time points after gavage (FIG. 15A and FIG. 15B). Blood tests further revealed an increase in red blood cell size (FIG. 15C and FIG. 15D) and a trend towards lower platelet count (FIGS. 15E and 15F) at 24 hours after gavage of PPACK NPs.

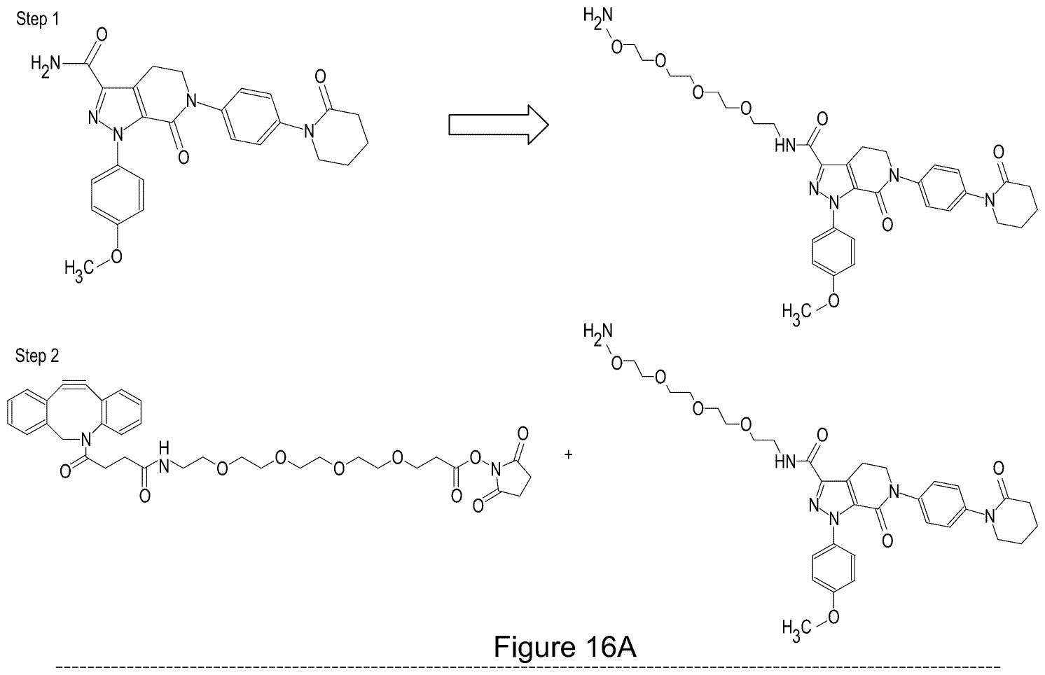

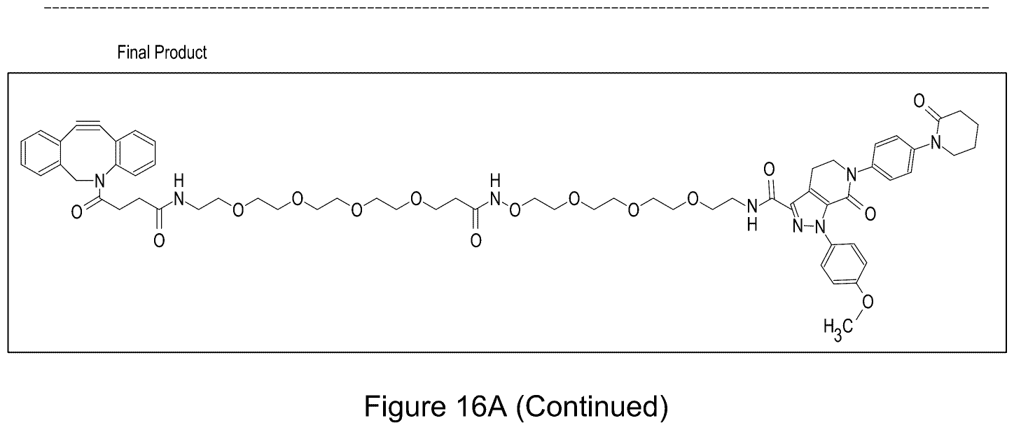

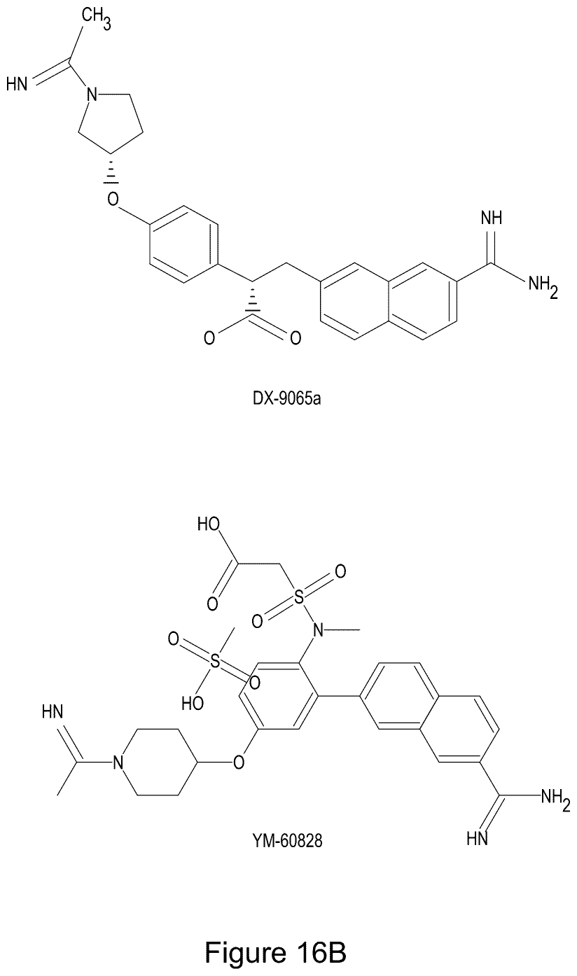

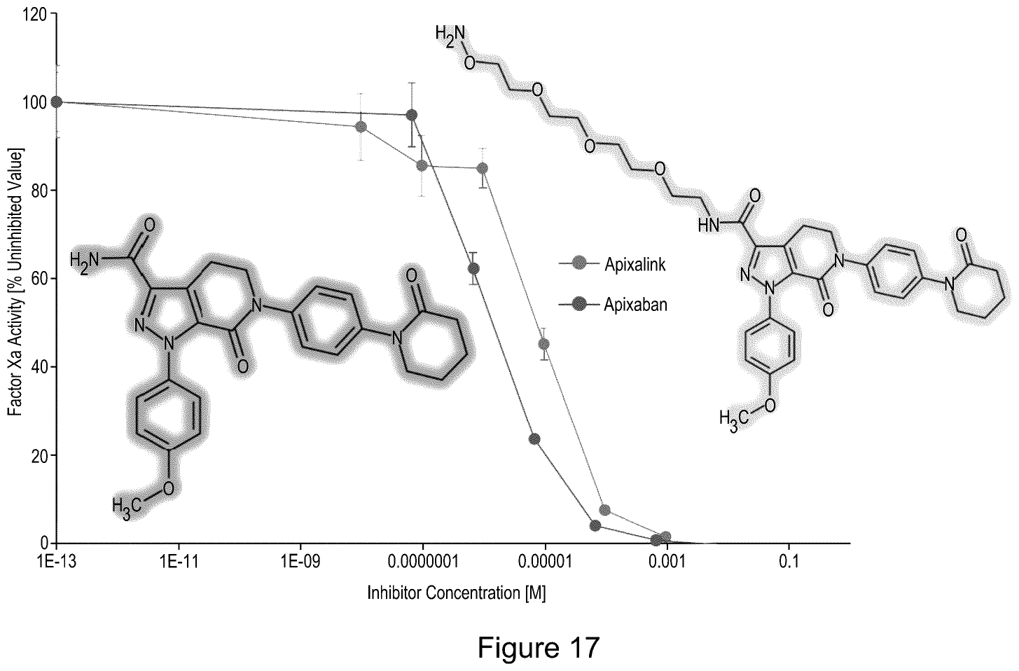

Demonstrating that different antithrombotics could be used in LTATs, a method was devised to modify the Factor Xa inhibitor, apixaban, for conjugation to lipids (FIG. 16). The modified apixaban was shown to inhibit Factor Xa activity as well as the unmodified apixaban (FIG. 17).

### Discussion

Formulating anticoagulant molecules on lipids for oral delivery demonstrated an approach for focal anticoagulant effects. Anticoagulant molecules linked to lipids can be specifically delivered to the lymph fluid, where they remain active. At the same time, anticoagulant molecules linked to lipids are constructed to be cleared from unwanted tissues and the blood stream. Anticoagulant molecules that are initially delivered as a liposome are broken down after administration, where lipids are redistributed among reformed chylomicrons. Anticoagulant molecules can be formulated as liposomes, micelles or free lipid in an oil emulsion. The present example demonstrated the conjugation and formulation process and functionality for a PPACK-conjugated lipid formulated in a liposome, however, formulations including anticoagulant molecules with higher specificity for thrombin or Factor Xa are being developed. Anticoagulant molecules with low oral availability when not lipid bound and having a short half-life in the blood stream could provide a more specific lymphatic targeted effect. Preferred molecules included those having a free acid or amine to allow conjugation of the unmodified molecule to a lipid head group with a covalent bond to prevent dissociation.

### Methods

PPACK liposomes were prepared by; 1) synthesizing liposomes with bio-orthogonal conjugation handles, in this case, azide-terminated lipids; 2) conjugating PPACK to a linker molecule with a bio-orthogonal conjugation handle with compatibility for the liposomes. In this case, PPACK was combined with dibenzocyclooctyne (DBCO) with an N-hydroxysuccinimide terminated poly (ethylene) glycol (PEG) linker. The N-hydroxysuccinimide reacted with the primary amine terminus of PPACK to form PPACK-DBCO. Liposomes were prepared with dipalmitoylphosphatidylcholine (DPPC) and cholesterol, alongside 1,2-distearoyl-sn-glycero-3-phosphoethanolamine (DSPE) with an azide-terminate PEG linker, in a formulation previously demonstrated to form highly stable liposomes. Liposomes with azide groups were reacted with PPACK-DBCO overnight and PPACK-DBCO that did not conjugate to liposomes was removed from the formulation by centrifugal filtration or size-exclusion chromatography, to assure that the formulation contained no PPACK that was not conjugated to lipids.

To modify apixaban for conjugation to lipids, a derivative of apixaban with a carboxylic acid group was obtained and a PEG-(bis)amine homobifunctional linker was conjugated to the apixaban derivative by carbodiimide coupling with one amine terminus protected by fluorenylmethyloxycarbonyl (FMOC) group. After de-protection with trifluoroacetic acid, the modified apixaban was purified by high performance liquid chromatography with a reverse-phase column and the structure of the final molecule was verified with mass spectrometry and nuclear magnetic resonance.

Inhibition of thrombin by LTATs, either in vitro or in samples of chyle or blood from LTAT-treated mice, was assessed with chromogenic substrate assays, where a chromogenic substrate, such as chromozym TH, is cleaved by thrombin or Factor Xa, liberating a p-nitroaniline group to produce an optical absorbance signature that is quenched by a tosyl group prior to cleavage. In all chromogenic substrate assays described, a fixed quantity of substrate and active enzyme, either thrombin or Factor Xa, were added to the assay alongside different concentrations of inhibitors, either as unconjugated molecules or as LTATs, or alongside samples from mice receiving LTATs, containing unknown concentrations of inhibitors. Optical absorbance signature indicated the degree of substrate cleavage by the set concentration of enzyme and diminution of the optical absorbance signature indicated the quantity of inhibitor added to the assay.

Although the presently disclosed subject matter and its advantages have been described in detail, it should be understood that various changes, substitutions and alterations can be made herein without departing from the spirit and scope of the disclosed subject matter. Moreover, the scope of the present application is not intended to be limited to the particular embodiments of the process, machine, manufacture, and composition of matter, methods and processes described in the specification. As one of ordinary skill in the art will readily appreciate from the disclosed subject matter of the presently disclosed subject matter, processes, machines, manufacture, compositions of matter, or methods presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, or methods.

What is claimed is:

1. A lipid conjugate, comprising:
a. one or more lymphatic condition, disease or disorder treatment active molecules; and
b. a lipid or lipid-like molecule.

2. The lipid conjugate of claim 1, further comprising a linker region.

3. The lipid conjugate of claim 1, further comprises a lipid head group.

4. The lipid conjugate of claim 1, wherein the one or more lymphatic condition, disease or disorder treatment active molecules is an anticoagulant molecule.

5. The lipid conjugate of claim 4, wherein the anticoagulant molecule is an anti-thrombin, anti-Xa molecule, or thrombolytic agent.

6. The lipid conjugate of claim 4, wherein the anticoagulant molecule blocks thrombin or Xa enzymatic activity.

7. The lipid conjugate of claim 1, wherein the anti-thrombin molecule is hirudin, bivalirudin, ximelagatran, a derivative of dabigatran, or a derivative of tripeptide-type thrombin inhibitors.

8. The lipid conjugate of claim 1, wherein the anti-Xa molecule is selected from the group consisting of Edoxaban, Rivaroxaban, Apixaban, or combinations thereof.

9. The lipid conjugate of claim 1, wherein the lipid comprises long chain fatty acids or monoglycerides.

10. The lipid conjugate of claim 1, wherein the lymphatic condition, disease or disorder active molecule is selected from an active agent that can treat the lymphatic condition, disease or disorder selected from the group consisting of sepsis, necrotizing enterocolitis, autoimmune diseases, Crohn's disease, celiac disease, ulcerative colitis, rheumatoid arthritis, cardiovascular disease, bacterial infections, viral infections, viral hepatitis (including hepatitis C virus hepatitis), alcoholic hepatitis, insulin resistance in adipocytes, pancreatitis, metabolic syndrome, a trauma induced inflammation, an acute respiratory distress syndrome (ARDS), a COVID-19-induced systemic inflammation, an organ rejection after transplantation, amyloidosis, lymphangitis, obesity, primary or secondary lymphedema, congenital lymphatic insufficiency, lymphatic vessel aplasia, inflammatory bowel disease, chronic granulomatous disease (CGD), lymphatic malignancies (including but not limited, Hodgkin's Disease, non-Hodgkin's lymphoma, and Castleman Disease), Milroy's disease, Meige's disease, elephantiasis, disorders of the lymphatic system arising secondarily to tissue damage, e.g., an infarction, injury from surgery, organ or tissue transplant, radiation therapy, chemotherapy, and occlusion or blockage (full or partial) of lymph vesselsm non-lymphatic malignancies, colorectal cancer, liver cancer, stomach cancers, pancreatic cancer, sepsis, necrotizing enterocolitis, autoimmune diseases, and Castleman Disease), Milroy's disease, Meige's disease, elephantiasis, disorders of the lymphatic system arising secondarily to tissue damage, e.g., an infarction, injury from surgery, organ or tissue transplant, radiation therapy, chemotherapy, and occlusion or blockage (full or partial) of lymph vessels.

11. A method of treating gut lymphatic thrombosis, comprising administering to a subject in need of such treatment, an effective amount of the lipid conjugate of claim 1, wherein administering an effective amount of lipid based conjugate preserves hemostasis within the subject.

12. The method of claim 11, wherein the lymphatic thrombosis is associated with an infection or inflammation in at least the gut.

13. The method of claim 11, wherein the lipid conjugate is selectively packaged with chylomicrons and transported from the gut in lymph.

14. The method of claim 11, wherein the lipid conjugate is not readily absorbed into the blood stream.

15. The method of claim 11, wherein the lipid conjugate is in the form of a tablet, a capsule, a sachet, a suppository, a liquid, an oil, or combination thereof.

16. The method of claim 11, wherein the lipid conjugate is administered orally.

17. The method of claim 11, wherein the lipid conjugate is a free lipid within an oil solution, micelle, liposomes, or solid lipid nanoparticles.

18. A method of preventing lymph clot formation using the lipid conjugate of claim 1.

19. A method for reducing inflammation of the gut or for treating infections of the gut using the lipid conjugate of claim 1.

20. A pharmaceutical composition comprising the lipid conjugate of claim 1.

21. A kit comprising the lipid conjugate of claim 1.
